(19) United States
(12) Patent Application Publication
(10) Pub. No.: US 2025/0331740 A1
(43) Pub. Date: Oct. 30, 2025

(54) MULTI-ANALYTE SENSING AND MEDICATION CONTROL

(71) Applicant: PercuSense, Inc., Valencia, CA (US)

(72) Inventors: Rajiv Shah, Rancho Palos Verdes, CA (US); Bradley Liang, Bloomfield Hills, MI (US); Brian Kannard, Los Angeles, CA (US); Katherine Wolfe, Dunwoody, GA (US); Blake Morrow, Los Angeles, CA (US); Kaitlynn Olczak, Valencia, CA (US)

(73) Assignee: PercuSense, Inc., Valencia, CA (US)

(21) Appl. No.: 19/040,781

(22) Filed: Jan. 29, 2025

Related U.S. Application Data
(63) Continuation-in-part of application No. 17/679,307, filed on Feb. 24, 2022.
Application No. 17/679,307 is a continuation of application No. 16/273,920, filed on Feb. 12, 2019, now Pat. No. 11,284,816.
(60) Provisional application No. 62/630,101, filed on Feb. 13, 2018.

(51) Int. Cl. A61B 5/145; A61B 5/1473; A61B 5/1486; A61B 5/1495

(52) U.S. Cl. CPC A61B 5/14532; A61B 5/14542; A61B 5/14546; A61B 5/1473; A61B 5/14503; A61B 5/14865; A61B 5/1495

(57) ABSTRACT

A multi-analyte sensor system is disclosed. The system includes a sensor probe that has a first set of electrodes that transduce glucose into electrical signals, a second set of electrodes that transduce lactate into electrical signals and a third set of electrodes that provide working and counter electrode functionality for the first and second set of electrodes. The system has an electronics module that electrically interfaces with the sensor probe, and includes a transceiver configured to transmit sensor data. The system also includes control circuitry communicatively coupled to the electronics module that determines a glucose state based on signals from the first set of electrodes and also determine a lactate state based on signals from the second set of electrodes. The control circuitry also generates an insulin infusion pump control signal based on signals from the first and second set of electrodes.

### RELATED APPLICATIONS

This application is a continuation-in-part of U.S. application Ser. No. 17/679,307, filed Feb. 24, 2022, which is a continuation of U.S. application Ser. No. 16/273,920, filed Feb. 12, 2019, which claims the benefit of U.S. Provisional Application No. 62/630,101, filed Feb. 13, 2018. The applications listed above are hereby incorporated by reference in their entireties for all purposes.

### BACKGROUND

The present disclosure generally relates to systems, devices, and methods for real time monitoring of physiological parameters to enable monitoring of physical conditions. More specifically, the present disclosure relates to the use of sensors and related control circuitry to enable at least partially automatic open-loop and/or closed-loop control of therapies associated with chronic conditions such as, but not limited to, diabetes.

It may be highly desirable to develop automatic insulin delivery (AID) systems that satisfactorily resolve glucose without, or at least minimizing the likelihood of, chronically high insulin. In some embodiments, the present disclosure provides solutions for the use of multianalyte sensors capable of detecting or measuring both glucose and lactate to help accomplish this goal. Examples of the present disclosure advantageously utilize real-time glucose and lactate measurements to resolve additional metabolic conditions that can help users not only be aware of their overall metabolic health, but also assist in balancing long term health goals for users of AID systems.

In some implementations, multiple analytes and/or physical parameters are monitored with respect to metabolic health and diabetes. While embodiments and examples discussed in detail below may be related to particular analytes and physical parameters, the scope of the disclosure and claims should not be construed to be limited to the specifically addressed analytes and parameters associated with metabolic health and diabetes. Rather, it should be recognized that additional/other analytes and/or physical parameters can be monitored to assist in the detection and diagnosis of various conditions or general physiological health.

### SUMMARY

In some implementations, the present disclosure relates to a multi-analyte sensor system is disclosed that includes a sensor probe. The sensor probe has a first set of electrodes with one or more first working electrodes configured to transduce glucose into electrical signals. The sensor probe also has a second set of electrodes with one or more second working electrodes configured to transduce lactate into electrical signals. The sensor probe further includes a third set of electrodes with one or more third electrodes that provide working and counter electrode functionality for the first set of electrodes and the second set of electrodes. The system has an electronics module that electrically interfaces with the sensor probe, the electronics module including a transceiver configured to transmit sensor data. Additionally included is control circuitry communicatively coupled to the electronics module. The control circuitry is configured to determine a glucose state based on one or more signals from the first set of electrodes and also determine a lactate state based on one or more signals from the second set of electrodes. The control circuitry is also configured to generate an insulin infusion pump control signal based on the one or more signals from the first set of electrodes and the one or more signals from the second set of electrodes.

The control circuitry can be a component of the electronics module. In some implementations, the control circuitry is further configured to determine a plasma insulin condition based on the one or more signals from the first set of electrodes and the one or more signals from the second set of electrodes, and the insulin infusion pump control signal is based on the plasma insulin condition. In some implementations, the control circuitry is further configured to determine an insulin sensitivity condition based on the one or more signals from the first set of electrodes and the one or more signals from the second set of electrodes, and the insulin infusion pump control signal is based on the insulin sensitivity condition. In some implementations, the sensor probe further comprises a fourth set of electrodes including one or more third working electrodes configured to transduce tissue oxygen into electrical signals, and the insulin infusion pump control signal is based on one or more signals from the fourth set of electrodes. In some implementations, the control circuitry is further configured to detect a meal intake state based on the glucose state, and the insulin infusion pump control signal is based on the detected meal intake state and directs a bolus insulin dose. In some implementations, the control circuitry is further configured to detect an exercise state based on the lactate state, and the insulin infusion pump control signal is based on the detected exercise state and directs reduction in insulin delivery to prevent a hypoglycemia state. In some implementations, the sensor probe is configured to detect tissue impedance, and the insulin infusion pump control signal is based on the detected tissue impedance. The multi-analyte sensor system can further comprise an accelerometer associated with the electronics module, wherein the insulin infusion pump control signal is based on one or more signals from the accelerometer that indicate physical activity.

In another embodiment, a continuous multianalyte monitoring system is disclosed that has a skin-mounted sensor control unit that includes a percutaneous multianalyte sensor with an insertion portion configured for transcutaneous positioning in a subcutaneous tissue of a user. The percutaneous multianalyte sensor is configured to sense levels of glucose and lactate in the subcutaneous tissue of the user. An adhesive patch disposed on a bottom surface of the skin-mounted sensor control unit is configured to adhere the skin-mounted sensor control unit to skin of the user. The skin-mounted sensor control unit further includes a transceiver for wireless communication with the skin-mounted sensor control unit. The skin-mounted sensor control unit has control circuitry that receives and stores signals from the percutaneous multianalyte sensor related to sensed levels of glucose and lactate. The control circuitry also determines a real-time insulin condition value based on the received signals of glucose and lactate and further determines a metabolic health score based on the real-time insulin condition value. Additionally, the control circuitry generates user interface data that is rendered on a touch-interface display to visually display a graph of the glucose and lactate levels. Where the graph represents a first axis corresponding to time, a second axis corresponding to one or more of the glucose levels or lactate levels, and the metabolic health score.

The control circuitry can be further configured to maintain historical real-time insulin condition values in data storage of the control circuitry. In some implementations, the continuous multianalyte monitoring system further comprises an automatic insulin delivery system configured to deliver basal insulin doses and bolus insulin doses based on at least one of the real-time insulin condition value or one or more of the maintained real-time insulin condition values. In some implementations, the control circuitry is further configured to determine a total insulin delivered by the automatic insulin delivery system based on basal insulin and bolus insulin delivered over a defined time period and determine a real-time insulin budget residual based on the total insulin. The real-time insulin condition value can indicate a plasma insulin level of the user and/or an insulin resistance condition of the user.

In another embodiment, a multi-analyte sensor system is disclosed that includes a sensor probe. The sensor probe has a first set of working electrodes configured to transduce glucose into first electrical signals. The sensor probe also has a second set of working electrodes configured to transduce lactate into second electrical signals. The sensor probe also has a third set of electrodes that are configured to provide reference and counter electrode functionality for the first set of working electrodes and the second set of working electrodes. The system further includes an electronics module configured to electrically interface with the sensor probe. The electronics module includes a transceiver configured to wirelessly transmit sensor data. Additionally included with the system is control circuitry communicatively coupled to the electronics module. The control circuitry is configured to store an insulin budget value that indicates an insulin budget for administration to a user over a set period of time. The control circuitry is further configured to determine a glucose state based on one or more signals from the first set of working electrodes and also determine a lactate state based on one or more signals from the second set of working electrodes. The control circuitry also is configured to determine an insulin condition based on at least the glucose state and the lactate state and determine an acute dose of insulin modification based on the insulin condition and an anticipated modified glucose state. The control circuitry additionally is configured to provide recommendations for the insulin modification to reduce the acute dose based on a residual of the insulin budget.

The insulin modification can be a change in basal insulin delivery. For example, the change in basal insulin delivery can be a suspension of basal insulin delivery. In some implementations, the insulin modification is an acute dose of insulin and the recommendations to reduce the acute dose is provided if the acute dose of insulin exceeds a projected residual of the insulin budget. The recommendations can include insulin dependent and non-insulin dependent recommendations to reduce the acute dose. For example, the insulin dependent recommendations can provide an option to favor closer adherence to the insulin budget rather than a recommendation to improve glucose time-in-range, and/or an option to favor close adherence to glucose time-in-range and exceeds the insulin budget. Non-insulin dependent recommendations can include behavior modifications that (i) reduce anticipated meal metrics that require insulin, or (ii) improve glucose clearance, or (iii) slow glucose absorption.

Other features and advantages of the invention will become apparent from the following detailed description, taken in conjunction with the accompanying drawings that illustrate, by way of example, various features of embodiments of the invention.

Methods and structures disclosed herein for treating a user/patient also cover analogous methods and structures performed on, or placed on, a simulated patient, which can be useful, for example, for training, demonstration, procedure and/or device development, and the like. For example, a simulated patient can be physical, virtual, or a combination of physical and virtual. A simulation can include a simulation of all or a portion of a patient, such as an entire body, a portion of a body, a system, an organ, or any combination thereof. Physical elements can be natural, including human or animal cadavers, or portions thereof; synthetic; or any combination of natural and synthetic. Virtual elements can be entirely in silicon, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

FIG. 1 is an exemplary block diagram showing components within a system configured to detect and process signals and/or data indicative of at least one physiological state of a subject, such as fasting, exercise, or postprandial conditions, and control or direct insulin infusion based thereon, in accordance with embodiments of the invention.

FIG. 2 is an exemplary block diagram of a combined device that integrates into a single device both the previously discussed sensor system 102 and medication dispenser.

FIGS. 3A-3D are exemplary block diagrams illustrating various electrode configurations of analyte sensor probes, in accordance with various embodiments of the present invention.

FIGS. 4A and 4B are exemplary views of an A-side and a B-side of an implantable probe, also referred to as an analyte sensor or sensor probe, that includes the sensor array, in accordance with embodiments of the invention.

FIG. 4C is an exemplary cross-section illustration of the analyte sensor illustrating diffusion of analyte, reactant and reaction by-product within one working electrode in accordance with embodiments of the present invention.

FIGS. 5A and 5B are an exemplary illustration of A-side and B-side of a sensor probe, in accordance with other embodiments of the present invention.

FIGS. 6A and 6B are an exemplary illustration of A-side and B-side of a sensor, in accordance with another embodiment of the present invention.

FIG. 7 is an exemplary illustration of real-time glucose, lactate, oxygen, and ROS data for various physiological states such as sleep, exercise, a meal, and stress, in accordance with embodiments of the present invention.

FIG. 8 is an exemplary flowchart illustrating one embodiment of detecting a physiological condition, in accordance with various embodiments of the present invention.

FIG. 9 is an exemplary block diagram illustrating interactions between the different components within the system to enable insulin delivery, in accordance with embodiments of the present invention.

FIGS. 10-1 and 10-2 are exemplary hybrid block/flow diagram that illustrates interactions between various components within the system in order to deliver insulin, in accordance with embodiments of the present invention.

FIG. 11 is an exemplary illustration of an insulin budget residual and analyte values with respect to time, in accordance with embodiments of the present invention.

FIG. 12 is an exemplary illustration of multiple days of historical insulin budget data, in accordance with embodiments of the present invention.

### DETAILED DESCRIPTION

Awareness of the importance of metabolic health is growing as its association with chronic conditions such as type 2 diabetes and heart disease becomes better understood. While continuous glucose monitoring (CGM) can provide some insight regarding metabolic health, monitoring additional metabolic analytes, such as lactate, can provide additional insight. For example, metabolic health scores/metrics can be determined based on lactate measurements, as relatively poor mitochondrial health can be associated with increased resting lactate generation and/or poor lactate clearance. Additionally, monitoring glucose and lactate can help address the growing healthcare crisis associated with diabetes that presently affects nearly 30 million people in the United States. Approximately 10 percent of those affected require intensive glucose and insulin management. In hospital patients, hypoglycemia in both diabetic and non-diabetic patients is associated with increased cost and short- and long-term mortality.

To prevent complications, diabetes generally requires ongoing management. CGM has been shown in studies to provide an effective way to improve glucose control, whether used with insulin injections or a continuous insulin pump. Certain closed-loop solutions are challenged by everyday situations where insulin requirements change rapidly and often unpredictably. Augmenting CGM with other analytes such as lactate can help identify behaviors associated with physiological states or conditions which can enable insight into, and even predict changes in, glucose and insulin dynamics that impact insulin delivery decisions. This insight can enable improved automated or personalized solutions that result in better control and less burden, particularly for users of automatic or automated insulin delivery (AID) systems.

Certain AID systems are capable of driving glucose within a subject to hypoglycemia. However, because AID systems generally cannot deliver glucagon, an AID system may necessarily drive glucose down relatively slowly to account for the system's inability to drive glucose up. Accordingly, some AID systems are configured to determine a quantity of “active insulin,” or “insulin on board,” that has been delivered by the AID system. Metrics of AID system therapy efficacy can include time-in-range of glucose and hemoglobin A1c (HbA1C) measurement, where lower HbA1C is generally considered better. However, in some instances, achieving these goals can lead to over delivery of insulin. Chronic over-delivery of insulin can lead to chronically high insulin, which can promote or exacerbate insulin resistance.

Automated insulin delivery (AID) technology can improve glucose control. In some implementations, examples of the present disclosure relate to AID solutions that advantageously provide automated closed-loop control, which can provide benefits of certain other solutions. For example, some AID solutions deliver only basal insulin and use conservative glucose targets that allow elevated HbA1c values without eliminating hypoglycemia. Certain artificial pancreas (AP) solutions control both basal and bolus insulin delivery and strive to achieve an HbA1c of less than 7% without significant hypoglycemia for nonpregnant adults. However, in the quest to achieve a desirable HbA1c, many AID and AP systems overdeliver insulin, which, as a chronic condition, can promote or exacerbate insulin resistance. Accordingly, example systems of the present disclosure that control both basal and bolus insulin delivery, balance glucose control goals, and/or minimize the likelihood of developing resistance to exogenous insulin provide substantial improvements over other solutions.

Metabolic health of a user can influence determinations of both basal and bolus insulin delivery. Examples of the present disclosure advantageously account for a user's metabolic health by incorporating additional real-time signals beyond glucose. For example, when combined with glucose, lactate signals can provide useful insights into secondary factors, such as metabolic stress or insulin resistance. Integration of metabolic health data can facilitate more personalized and adaptive glucose control.

The determination of secondary conditions, particularly those associated with metabolic conditions, may depend upon many factors that influence a person's metabolism, including demographic and personal health information. Exemplary demographic data that can influence a secondary condition, such as an insulin condition, include, but are not limited to, age and sex. Personal health information that may be used in connection with embodiments disclosed herein to determine a secondary condition includes, but is not limited to, measures or metrics of adiposity or visceral fat, such as waist circumference, body mass index (BMI), data from a DEXA scan, or a bioimpedance measurement. Additional personal health information related to examples of the present disclosure includes systolic and/or diastolic blood pressure, and levels of adiponectin, cholesterol and triglycerides. Additional personal health information may be related to both acute and chronic conditions of a subject. With respect to the measurement of metabolic analytes, conditions that affect metabolic analytes may be of interest. Exemplary conditions that can impact or change the determination of a secondary condition include, but are not limited to, cancer, high blood pressure, type 1 or type 2 diabetes, chronic obstructive pulmonary disease, non-alcoholic fatty liver disease, and the like.

The determination of glucose, lactate and secondary conditions associated with them can further be influenced by specific behaviors such as meals, exercise, stress, medication, sleep, and special diets. In some implementations, examples of the present disclosure account for when specific or discrete behaviors are performed by a user to transform or correct glucose and/or lactate data. The objective of transforming or correcting the glucose and/or lactate data can advantageously ensure that a secondary condition, such as an insulin condition that is based on the measured or detected analyte levels, remains representative of actual conditions within the subject.

Embodiments disclosed herein enable the balancing of blood glucose control relative to insulin delivery based at least in part on glucose, lactate and secondary conditions that may be impacted by physiological conditions such as, but not limited to, meals, exercise, stress, and sleep. In some embodiments, the detection of physiological conditions is accomplished using a combination of biochemical signals associated with glucose and lactate, along with signals from physical sensors. The biochemical signals and optional physical signals can be derived from the same minimally-invasive probe used to produce a continuous glucose signal without increasing implant size. The ability to measure multiple biochemical signals via a single probe, combined with optional physical sensors in the same package results in a system that reduces burden on the subject rather than requiring mindfulness of multiple sensor insertions and separate physical sensors. The seamless integration of multiple signal streams can enable 24/7 insulin delivery automation. Such integration can further enable rapid individualization optimization efforts from the additional time series data generated and the data that can be distilled from the interaction between signal streams. It should be noted that removal of an insulin delivery device from the system results in a multianalyte sensor system that can provide actionable metabolic health data to a user. Accordingly, while much of the discussion below is related to delivery of exogenous insulin via an AID system, subjects that do not require exogenous insulin can benefit from such systems via actionable recommendations to improve their overall metabolic health based on real-time measurements of glucose, lactate, and/or secondary conditions.

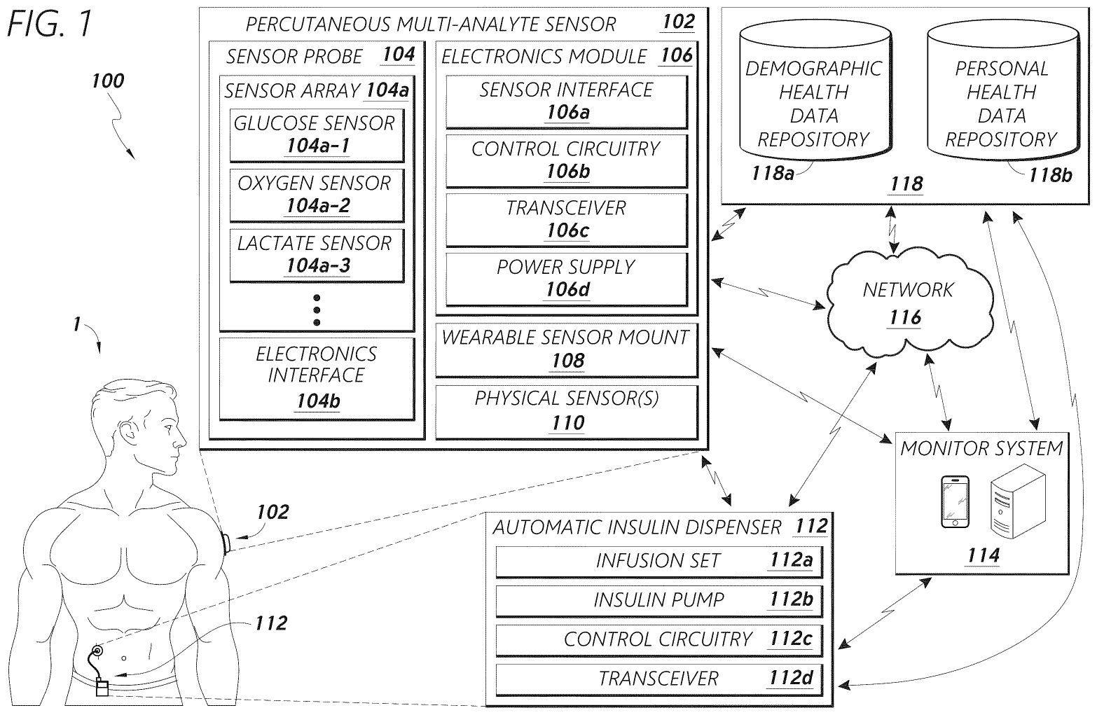

FIG. 1 is an exemplary block diagram showing components of a system 100 configured to detect and process signals and/or data sets indicative of at least one physiological state of a subject 1 (e.g., fasting, exercise, or postprandial conditions), and automatically control or direct insulin infusion based thereon, in accordance with embodiments of the invention. The system 100 advantageously provides a technical improvement for analyte sensor and medication delivery systems by integrating multiple analyte-sensing and data-processing functions that allow real-time insulin dosing or dosing recommendations to be made with heightened accuracy and reliability. Broadly, the system 100 includes a percutaneous multi-analyte sensor system 102 that includes a sensor probe 104 that is electrically coupled to an electronics module 106 via an electronics interface 104b. The sensor probe 104 advantageously is configured to capture, when implanted in a transcutaneous/percutaneous position inserted at least partially into subcutaneous tissue of the user 1, multiple analyte signals (e.g., glucose, lactate, oxygen) at a single insertion site, thereby reducing patient discomfort compared to multiple separate sensors. Optionally, a sensor mount 108 and one or more physical sensors 110 (e.g., accelerometers, thermometers) may be included within the sensor system 102. The sensor mount may be a skin-mounted/mountable unit to which the sensor probe 104 and/or electronics module 106 is/are physically coupled. Collectively, these components provide a hardware-based platform capable of continuous, real-time measurements for improved metabolic state determinations.

In preferred embodiments, the analyte sensor probe 104 is an electrochemical sensor probe that includes a sensor array 104a configured to measure/detect specific molecules of interest in vivo. Using specialized electrode configurations, the sensor array 104a can implement electrochemical sensing to simultaneously measure concentrations of glucose, lactate, and/or one or more additional analytes. For example, a glucose sensor 104a-1 of the sensor array 104 can be configured to implement amperometric detection with a selective enzyme coating of glucose oxidase, whereas a lactate sensor 104a-3 can be configured to implement lactate oxidase for specificity. This approach advantageously leverages real-time biochemical measurements to determine dynamic physiological states, such as metabolic stress during exercise or insulin sensitivity during fasting, and can significantly enhance the control of insulin dosing or other medication deliveries.

In some embodiments, the sensor array 104a further includes the capability or option to detect or measure an optional third analyte of molecule of interest. For example, as illustrated in FIG. 1, the sensor array 104a includes an optional oxygen sensor 104a-2. The illustration in FIG. 1 of the glucose sensor 104a-1, the lactate sensor 104a-3, and the oxygen sensor 104a-2 should not be construed as limiting. In some embodiments, the sensor array 104a can include additional sensors to detect or measure other molecules or analytes of interest such as, but not limited to ketone sensors using potentiometric methods, reactive oxygen species (ROS) sensors using chronoamperometry, and/or sensors to detect/measure choline, acetylcholine, alcohol and/or the like. Any such additional analyte sensors can be integrated to further enhance detection of metabolic conditions such as ketosis or oxidative stress. The incorporation of three or more sensor channels in a single device enables improved accuracy in detecting and predicting metabolic shifts, supporting advanced feedback-based dosing algorithms and mitigating risks associated with hypo- or hyperglycemic events.

The electronics interface 104b facilitates electrical communication between the analyte sensor probe 104 and the electronics module 106. While illustrated as part of the sensor probe 104, in other embodiments the electronics interface 104b may be embodied at least in part in the electronics module 106. As the electronics interface 104b is intended to interface between the analyte sensor probe 104 and the electronics module 106, its relative association or location between the elements or components within the sensor system 102 should not be construed as limiting.

In some embodiments, the electronics module 106 includes a sensor interface 106a, a communication module (e.g., transceiver configured to transmit sensor date) 106c, and/or a power supply 106d. In some implementations, the sensor interface 106a is configured to enable electrical coupling between the electronics module 106 and the electronics interface 104b. The sensor interface 106a can be configured to enable electrical signals generated by the analyte sensor probe 104 to be transmitted to the control circuitry 106b.

The electronics module 106 further includes additional control circuitry 106b in addition to the sensor interface 106a, communications/transceiver circuitry 106c, and power supply circuitry 106d, wherein the control circuitry 106b may be configured to perform certain signal processing, amplification, filtering, conversion, calibration, and management/control functions for the sensor system 102. In preferred embodiments the control circuitry 106b may include, but is not limited to elements such as clocks, memory, processors, analog-to-digital converters and the like. Such components can enable real-time signal processing, including filtering, amplification, and transformation of raw electrochemical signals into calibrated glucose and lactate concentrations. For example, the processor applies adaptive algorithms to correct for temperature variations or cross-analyte interference, ensuring accurate real-time data for physiological state determination. By integrating these functions within a single hardware platform, the system 100 offers enhanced reliability and responsiveness, supporting improved safety and efficacy in automatic insulin or medication delivery.

The control circuitry 106b may be configured to enable control of the analyte sensor probe 104. The control circuitry 106b can further enable data processing of signals generated or detected by the analyte sensor probe 104. For example, the control circuitry 106b can be configured to apply machine-learning models trained on personal, demographic, and/or historical data to dynamically adjust insulin dosing/recommendations based on detected glucose-lactate trends. For example, in some embodiments, the control circuitry 106b enables transformation of raw signals from the analyte sensor probe 104 to be representative of the respective molecule or analyte being detected. The control circuitry 106b can further generate and/or display information that is more meaningful for the subject than analyte or molecular concentrations. The terms “circuitry” and “control circuitry” are used herein according to their broad and ordinary meanings, and may refer to any individual or collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

Further included in the electronics module 106 is a communication module (e.g., transceiver) 106c. The communication module 106c enables communication between the sensor system 102 and other components within the system 100. In many embodiments the communication module 106 enables two-way communication via any suitable or desirable communication protocol(s), such as, but not limited to Wi-Fi, Bluetooth or wireless network technologies such as 4G, 5G and the like. The communication module 106c enables data acquired by the sensor system 102 to be transmitted in either raw, partially processed, or fully processed to other components within the system 100. Additionally, the communication module 106c further enables data from other components within the system 100 to be used as input for the sensor system 102. For example, in many embodiments the communication module 106c enables data from an electronic health record to be dynamically input into the control circuitry of the sensor system 102. A power supply 106d is also included as part of the electronics module 106. In preferred embodiments the power supply 106d is an energy storage device such as a disposable or rechargeable battery. In alternative embodiments the power supply 106d may include or be based on energy storage technologies such as, but not limited to solar cells, capacitors, fuel cells and the like.

In some embodiments, the sensor system 102 optionally includes a sensor mount 108, which may be attached or coupled to the subject/user 1 (e.g., using an adhesive patch disposed on a bottom surface of the skin-mountable mount 108) and is configured to receive and secure the electronics module 106. In many embodiments, the sensor probe 104 may be coupled or attached to the sensor mount 108. Insertion of the sensor probe 104 may serve to couple the sensor mount 108 to the subject. After insertion of the sensor probe 104, the electronics module 106 may be coupled to the sensor mount 108 and begin providing power to the sensor probe 104. In some embodiments, the electronics module 106 may be removably coupled to the sensor mount 108, such that the electronics module 106 may be reusable in its entirety, which may be particularly advantageous for embodiments that utilize a rechargeable or replaceable power supply. In other embodiments, portions of the electronics module 106 may be reused or recycled to reduce overall electronic waste.

In many embodiments, one or more physical sensors 110 may be optionally included as part of the sensor system 102. The inclusion of physical sensor(s) 110 can enable detection of parameters that can affect the integrity or validity of data acquired via the sensor probe 104. Exemplary, non-limiting physical sensors that may be integrated within the sensor system 102 include, but are not limited to, accelerometers, thermometers and the like, which can provide improvements in insulin management technologies by enhancing system accuracy through detection and response to external conditions that influence analyte levels. For instance, accelerometer data can indicate periods of sustained motion corresponding to exercise or movement, allowing the system to adjust lactate-derived insulin dosing thresholds in real time. For example, because exercise can affect both glucose and lactate concentrations within the subject, the inclusion of physical sensors 110 capable of detecting movement enables sustained motion such as exercise to be used as an input to modify or control other aspects of the system 100.

The system 100 can additionally include a medication dispenser 112 configured to dispense precise bolus and basal doses of insulin, or any other type of medication, therapeutic agent, drug, treatment, delivery agent, or other therapeutic substance. An exemplary, non-limiting medication dispenser 112 is a portable infusion pump, such as an insulin pump or insulin pen. In some embodiments, the medication dispenser 112 may have the capability to automatically dispense medication such as an automatic insulin delivery (AID) system. In some embodiments, the medication dispenser 112 may comprise a smart insulin pen. As illustrated in FIG. 1, the medication dispenser 112 is an AID system that includes an infusion set 112a, an infusion pump 112b, control circuitry 112c, and/or a transceiver 112d.

The system 100 further includes a data repository 118. The data repository 118 can advantageously store data that can influence or have an impact on data provided by the sensor system 102 and/or the medication dispenser 112. Exemplary data retained in the data repository 118 can include, but is not limited to, data indicating attributes of the subject 1 and/or demographic population(s) relating age, gender, height, weight, body mass index, waist circumference, blood pressure (diastolic or systolic), cholesterol, any and quantities of both chronic and acute medications, along with any chronic conditions. The exemplary data described above that can be stored in the data repository 118 should not be construed as limiting. In preferred embodiments, any type of health metric that may be recorded in an electronic or physical health record may be input and stored in the data repository 118. The demographic and/or personal health data can enable the system to contextualize real-time sensor outputs, such as correlating lactate trends with metabolic health conditions like insulin resistance or non-alcoholic fatty liver disease, to refine insulin delivery algorithms dynamically.

In many embodiments, the data repository 118 includes a demographic data repository 118a and a personal data repository 118b. In some implementations, the demographic health data 118a and the personal health data 118b are stored in the same physical data storage device(s) or server(s). Both the demographic data repository 118a and the personal data repository 118b may store data of a similar type. However, in preferred embodiments, the demographic data repository 118a is anonymized, while the personal data repository 118b is specific to a particular user or subject. The demographic data repository 118b can enable analysis of data across various demographics represented by the data stored therein. For example, in some embodiments, the demographic data repository 118a enables artificial intelligence or other trainable model configured to analyze the data for patterns or trends that can be applied to modify or control other components within the system 100.

A network 116 is included within the system 100 to enable communication between various components within the system 100. The network 116 may leverage various communication protocol(s), such as cellular or mobile networks (e.g., 5G, 4G and the like), Wi-Fi, Bluetooth, Zigbee and/or the like. The network 116 can enable data from either or both of the sensor system 102 or the medication dispenser 112 to be stored in the data repository 118. Additionally, the network 116 can enable the use of data stored in the demographic data repository 118a and/or personal health data repository 118b as input to control or modify control of other components within the system 100.

The system 100 further includes a monitoring system 114, which may be local, remote, or both. In preferred embodiments, the monitor system 114 leverages the network 116 to communicate with other components within the system 100. In some embodiments, the monitor system 114 includes the ability to process data from the various components within the system 100. For example, in some embodiments the monitor system 114 can receive data from the sensor system 102 as raw data and process the raw data to be representative of the respective analytes or molecules. In some embodiments, the monitor system 114 is configured to receive processed data from other components within the system 100. In some such embodiments, the monitor system 114 can supplement the processed data with data from other components and further transform the data.

Transformation of data from the sensor system 102 enables determination of secondary considerations or conditions based on, or derived from, real-time measurements from the sensor system 102. “Secondary conditions,” as described herein, may be any physiological or metabolic states that are derived from or influenced by primary data (e.g., glucose levels, lactate levels, and/or oxygen levels) measured by a sensor (e.g., multi-analyte sensor) of a system. Secondary conditions can comprise higher-order conditions inferred from the integration of primary analyte data with other inputs (e.g., activity, medication, chronic health status), and can provide insights into the subject's metabolic health and/or guide adjustments to insulin delivery or other therapeutic interventions. Secondary conditions can include data structures representing any of insulin resistance, metabolic stress, fasting state, postprandial state, chronic disease impact, hypoxic or oxygen-deprived states, medication interactions, or the like.

Exemplary data stored in the data repository 118 that may be used to transform sensor system 102 data to a secondary condition include factors, states, or conditions that influence metabolic health. For example, a measure of adiposity or visceral fat such as, but not limited to, waist circumference, body mass index, a bioimpedance measurement or data from a dual-energy X-ray absorptiometry (DEXA) scan. Additional metabolic health influencing factors that may be obtained from the data repository 118 include, but are not limited to, age, gender, diastolic and/or systolic blood pressure, cholesterol levels, triglyceride levels and adiponectin levels. Additional inputs or factors that may be stored and retrieved from the data repository 118 that can be used to determine a secondary condition include information regarding any chronic disease states or treatment thereof that can influence or alter the metabolic health of a subject. Non-limiting, exemplary chronic conditions include, but are not limited to, high blood pressure, cancer, chronic obstructed pulmonary disease, whether a subject has type 1 or type 2 diabetes, and whether a subject has been diagnosed with non-alcoholic fatty liver disease (NAFLD). In addition to the presence or status of a chronic condition or medical diagnosis, in some embodiments, the data repository 118 can further include data regarding any medications and the respective doses the subject is taking for the chronic condition.

In still further embodiments, dosing of particular medications, such as those associated with both type 1 and type 2 diabetes like metformin, GLP agonists, and insulin via multiple daily injections or via a portable infusion device and/or automatic insulin delivery device, and even their anticipated dosing times, may be used as an input to determine a secondary consideration based on data from the sensor system 102. In many preferred embodiments, an exemplary, non-limiting secondary condition that can be determined from various inputs to the system 100 is the determination of an insulin condition in real-time. In some embodiments, the insulin condition is associated with insulin resistance for a subject. Additionally or alternatively, the insulin condition can be associated with plasma insulin levels within a subject.

In some embodiments, the monitor system 114 includes a display. In preferred embodiments, the monitor system 114 displays data from various components of the system 100, such as analyte levels detected by the sensor system 102 or a quantity or volume of medication dispensed by the medication dispenser 112. Inputs from the data repository 118 can be processed by an artificial intelligence module within the system 100, which is configured to apply machine learning algorithms to analyze historical and real-time data from the sensor system 102, enabling predictive modeling that dynamically adjusts insulin delivery parameters and enhances the system's ability to prevent hypo- or hyperglycemia. In still other embodiments, based on predictive data, the system 100 can determine and display on the monitor system 114 recommendations to the user to improve predictive data relative to long or short term goals or objectives associated with the metrics from components within the system 100. Exemplary, non-limiting embodiments of the monitor system 114 include, but are not limited to systems such as mobile phones, tablets, laptop computers, desktop computers, vehicle infotainment systems, home automation systems, and the like.

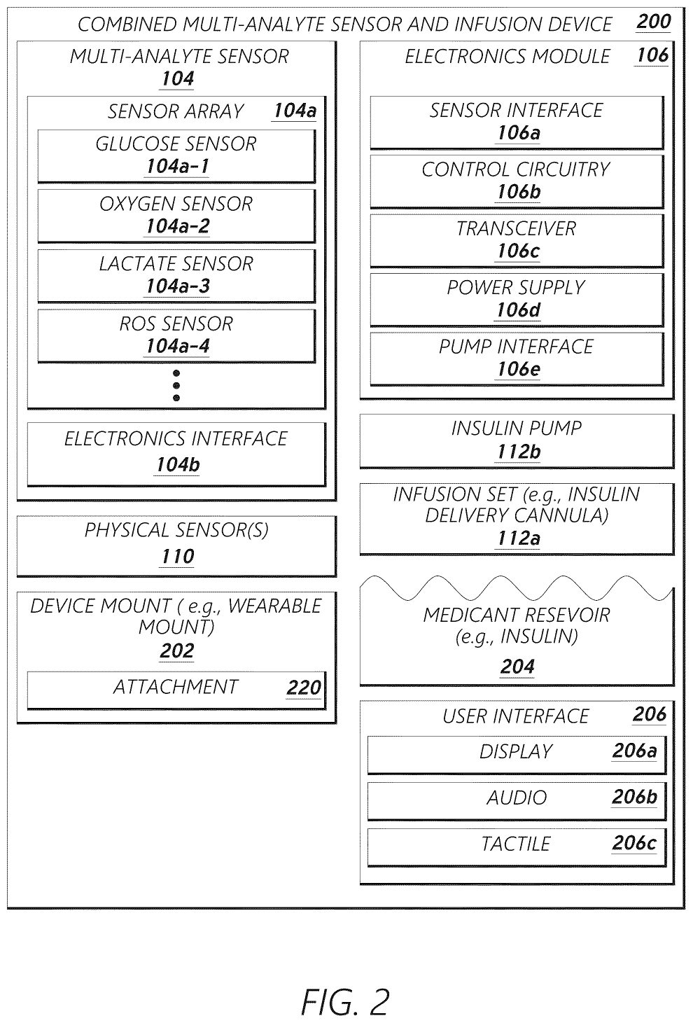

FIG. 2 is an exemplary block diagram of a combined device 200 that integrates into a single device both the previously discussed sensor system 102 and medication dispenser 112. With respect to inventive multi-analyte systems and processes disclosed herein, integration of the sensor and dispenser components can provide technical improvements with respect to the technical challenge of simultaneously monitoring real-time analyte levels and delivering precise medication doses through coordinated operation of the sensor system 102 and infusion pump 112b, and further provides technical improvements with respect to reduced insertion sites, simplified user operation, and improved data fidelity from co-located sensors.

The combined device 200 includes the analyte sensor probe 104 having the sensor array 104a. Similar to FIG. 1, the sensor array 104a may include the glucose sensor 104a-1, the oxygen sensor 104a-2, and/or the lactate sensor 104a-3. The specific analytes described above and illustrated within the sensor array 104a should not be construed as limiting. The sensor array 104a of the analyte sensor probe 104 may be configured to detect or measure more or fewer analytes of interest. Moreover, the sensor array 104 may be configured to detect or measure additional or different analytes than those described herein. For example, in some embodiments, any molecule capable of being detected or measured electrochemically may be implemented within the sensor array 104a.

The sensor array 104a includes the electronics interface 104b that enables the analyte sensor probe 104 to be coupled with the electronics module 106. The combined device 200 can further include physical sensors 110, along with a device mount 108. The device mount 108 enables the combined device 200 to be removably mounted or applied to a subject. The combined device 200 additionally includes the electronics module 106 that has the sensor interface 106a, the control circuitry 106b, the communication module 106c, and the power supply 106d. The electronics module 106 can also include a pump interface 106e to control the infusion pump 112b. The pump interface 106e may provide motion control to ensure a proper amount of medication is delivered by the infusion pump 112b.

The infusion pump 112b can be configured to deliver medication to a subject via the infusion set 112a, which may comprise a delivery cannula and may be used to deliver insulin, for example. The infusion set 112a is in fluid communication with a medication reservoir 204. In many embodiments, the medication reservoir contains a medication such as, but not limited to, insulin. In some embodiments, the combined device 200 can include more than one medication reservoir 204, which can enable infusion of multiple medications by a single device. For example, in some embodiments the combined device 200 may be capable of infusing insulin and glucagon.

The combined device 200 may be removably coupled or secured to a subject using a device mount 202. In some embodiments, the device mount 202 includes a base to which the analyte sensor probe 104 is attached and placement of the device mount 202 on the subject coincides with insertion of the analyte sensor probe 104 within the subject. In such embodiments, addition of the remaining components to the device mount 202 can complete the combined device 200. Moreover, in such an embodiment, the infusion set 112a may be inserted separately from the sensor probe 104. In other embodiments, the combined device utilizes a single insertion to place both the analyte sensor probe 104 and the infusion set 112a within the subject.

The combined device 200 further includes a user interface 206. The user interface 206 may include one or more visual display components 206a, audible components 206b, and/or tactile components 206c. In some embodiments, the visual/display component(s) 206a can include one or more lights that indicate a status of the combined device. Similarly, the audio component(s) 206b may include one or more piezoelectric devices or other sound-emitting device configured to produce audible sounds or sequences of sounds to convey or indicate a status of the combined device 200. Likewise, the tactile component(s) 206 may include vibration devices that create a tactile sensation or sequence of tactile sensations to convey or indicate a status of the combined device 200.

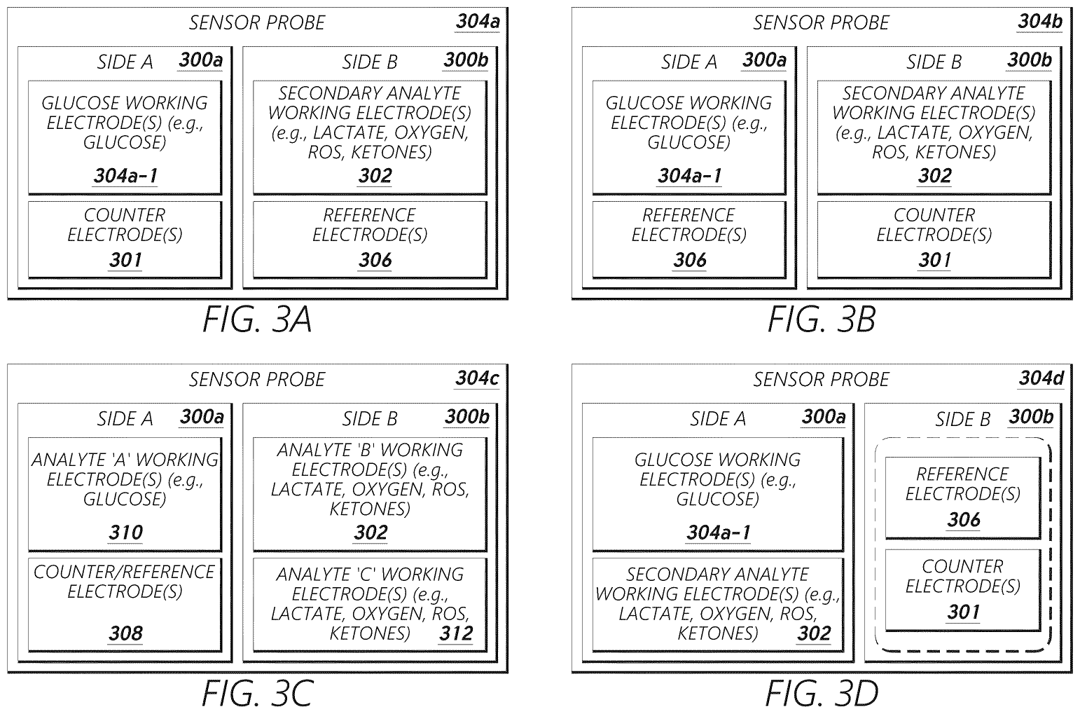

FIGS. 3A-3D are exemplary block diagrams illustrating various electrode configurations of analyte sensor probes 304a-304d, in accordance with various embodiments of the present disclosure. Example analyte sensor probes of the present disclosure may have configurations of any of the analyte sensor probes 304a-304d. The analyte sensor probes 304a-304d illustrated in FIGS. 3A-3D each include two sides/faces, namely an ‘A-side’ 300a and a ‘B-side’ 300b. For example, the sensor probes 304a-304d can have a thin, flat, elongated body with a generally rectangular cross-section, wherein the probes 304a-304d have two relatively broad, flat faces on opposite sides, which are parallel and define the primary surface area of the probe. Edges may generally run along the length of the probes 304a-304d where the two faces meet, forming two elongated, relatively narrow surfaces. Sides ‘A’ and ‘B’ of the sensor probes 304a-304d may correspond to the opposite-facing primary surfaces/faces of the respective probes. By implementing two-electrode or three-electrode systems (or hybrid configurations) on a single, thin, elongated sensor probe, these embodiments enable multi-analyte sensing while minimizing patient discomfort and maximizing measurement accuracy.

Each side of the analyte sensor probes 304a-304d may be configured with one or more electrodes, wherein each electrode can be configured as one of a working electrode, a counter electrode, a reference electrode, or a combined counter and reference electrode. In some embodiments, a two-electrode configuration is implemented, wherein a working electrode is operated with a combined counter and reference electrode. In other embodiments, a three-electrode configuration may be implemented, wherein a working electrode is operated with a separate counter electrode and a separate reference electrode. In some embodiments of a two-electrode system, multiple working electrodes operated with the same polarity may share a combined counter and reference electrode. Similarly, in some embodiments of a three-electrode system, multiple working electrodes operated with the same polarity may share a counter electrode and a reference electrode.

FIG. 3A is an exemplary illustration of a three-electrode sensor probe configuration 304a, wherein the A-side 300a includes a glucose working electrode 304a-1 and one or more counter electrodes 301. The B-side 300b is configured with a second working electrode 302 configured to measure or detect a second analyte (e.g., lactate, oxygen) and further includes one or more reference electrodes 306.

FIG. 3B is an exemplary illustration of a three-electrode sensor probe configuration 304b, wherein the A-side 300a includes a glucose working electrode 304a-1 and one or more reference electrodes 306. The B-side 300b is configured to measure or detect a second analyte 302 and further includes one or more counter electrodes 301.

FIG. 3C is an exemplary illustration of a two-electrode sensor probe configuration 304c, wherein the A-side 300a is configured with a first working electrode to measure a first analyte 310 and further includes a combined counter and reference electrode. The B-side 300b is configured with a second working electrode 302 and a third working electrode 312. In various embodiments, the second working electrode 302 may be configured to detect the same or a different analyte than the first working electrode. Similarly, the third working electrode 312 may be configured to detect the same or a different analyte than the first working electrode 310 and/or the second working electrode 302.

FIG. 3D is an exemplary illustration of a three-electrode sensor probe configuration 304d, wherein the A-side 300a includes a glucose working electrode 104a-1 and a second working electrode 302. The B-side 300b is configured with one or more reference electrodes 306 and one or more counter electrodes 304. The permutations illustrated in FIGS. 3A-3D and described above should be construed as exemplary rather than limiting. For example, in some embodiments a two-electrode system may be implemented on the same analyte sensor probe 104 as a three-electrode system. In such an embodiment the first and second working electrodes may be formed on the A-side while the B-side includes a combined counter and reference electrode along with a discrete counter electrode and a discrete reference electrode.

Any of the example configurations of FIGS. 3A-3D may be implemented in connection with embodiments of the present disclosure. In a given implementation, the positions of the electrodes may advantageously be optimized for accurate multi-analyte sensing by leveraging the geometry of the probe and the electrochemical requirements of each electrode. For example, the glucose electrode may desirably be positioned near the tip on the A-side, ensuring direct exposure to interstitial fluid in highly vascularized regions for consistent glucose measurements. The lactate electrode may also be positioned on the A-side, located slightly proximal to the glucose electrode, allowing it to measure lactate levels reflective of metabolic activity downstream from glucose uptake. An oxygen electrode, where present, may be positioned near the middle of the B-side of the probe, separated from the glucose and lactate electrodes to avoid interference from their electrochemical reactions while providing critical oxygen measurements that contextualize metabolic activity. The reference electrode may be placed centrally along the probe's B-side, such as equidistant from the working electrodes on both sides, to maintain a stable potential across all measurements. The counter electrode, where separate from the reference electrode, may be positioned at the proximal end of the B-side, providing uniform current flow for all working electrodes while minimizing interference with analyte detection. This example beneficial arrangement, and/or aspects or considerations associated therewith, can advantageously provide for reduced cross-interference, stable signal acquisition, and/or efficient use of the probe's surface area, enabling robust, synchronized multi-analyte sensing with a single implantable device.

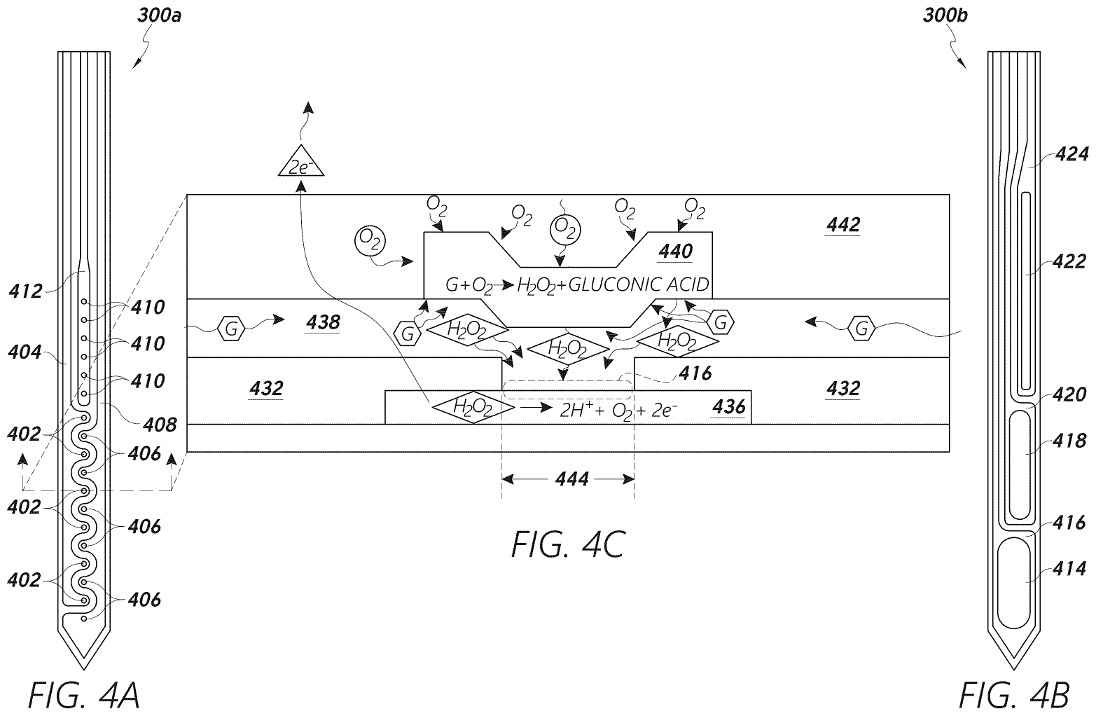

FIGS. 4A and 4B are exemplary views of an A-side 300a and a B-side 300b of an implantable probe, also referred to as an analyte sensor or sensor probe, that includes the sensor array 104a, in accordance with embodiments of the invention. In varying embodiments, the sensor array 104a includes an A-side 300a and a B-side 300b that enables continuous detection of glucose and at least a second analyte, such as lactate. While some embodiments of the analyte sensor probe 104 use both A-side 300a and B-side 300b, other embodiments utilize only a single side. In addition to glucose, exemplary second analytes that can be measured on the A-side 300a, B-side 300b, or across both A-side 300a and B-side 300b, include, but are not limited to, lactate, oxygen, reactive oxygen species (ROS), ketones, and the like. While illustrated as a single probe, varying embodiments of the sensor array 104a include multiple probes, each capable of measuring identical or different analytes using different combinations of working electrodes. Examples of a sensor array 104a having multiple probes but a single point of entry can be found in combined sensing and infusion devices discussed in U.S. patent application Ser. No. 15/455,115 filed on Mar. 9, 2017 which is hereby incorporated for reference for all purposes.

In some embodiments, the analyte sensor probe 104, or implantable probe, can be implanted via a surgical procedure. In other embodiments, the analyte sensor probe 104 can be temporarily inserted into tissue, such as, but not limited to subcutaneous tissue, muscle tissue, organ tissue, or the like. In some embodiments, the implantable probe 104 may be temporarily inserted into tissue for varying durations that can be measured in minutes, hours, days, weeks, or months. While many embodiments of the implantable probe, or analyte sensor, 104 have been discussed as using both an A-side 300a and B-side 300b, other embodiments utilize a single side of the implantable probe.

FIG. 4A is a view of the A-side 300a that includes first working electrodes 402 and second working electrodes 406 along with corresponding first electrode trace 404 and second electrode trace 408. In many embodiments the first working electrodes 402 are transducers configured to detect, or measure, glucose concentration. The second working electrodes 406 can be configured to measure the concentration of a second analyte such as, but not limited to lactate, oxygen, ROS, ketones, or the like. FIG. 4B is a view of the B-side 300b that includes a plurality of combination counter-reference electrodes 414 and 418 formed on electrode traces 416 and 420 respectively. In preferred embodiments a two-electrode system consisting of the first and second working electrodes with corresponding combined counter-reference electrodes, or pseudo-reference electrodes, are used to detect concentrations of the various analytes. However, other embodiments may use a three-electrode system having a working electrode along with a counter electrode and a reference electrode.

FIGS. 4A and 4B further include optional third working electrodes 410 formed on third electrode trace 412 in addition to third counter-reference electrode 422 formed on electrode trace 424. In some embodiments, the third working electrodes 410 can be duplicative of the second working electrodes 406, such that the third working electrodes 410 measure or detect the same analyte as the second working electrodes 406. However, in other embodiments, the third working electrodes 410 are used to measure a third analyte, such that the analyte sensor probe 104 (see FIG. 1) is capable of measuring glucose, and at least two of oxygen, lactate, ROS, ketones or the like. Common among the embodiments is at least measuring glucose because glucose measurements can assist in driving therapy decisions but can lack the ability to predict changing insulin dynamics. The addition of the second analyte and optional third analyte are intended to supplement glucose and provide the ability to predict changing insulin dynamics.

Measurement of some physical characteristics can be enabled via physical sensors or other instrumentation incorporated within or on the electronics module. With further reference to FIG. 1, in many embodiments, detecting a physiological state is accomplished via a combination of the analyte sensors 104 implanted within the subject and the physical sensors 110 associated with the electronics module 106. The specific physical sensors 110 discussed should not be construed as limiting. Other and additional physical characteristics from physical sensors 110 associated with the sensor system 102 can be used as inputs to detect or confirm various physiological states. Monitoring hydration levels of at least one, some, or all of the transducers within the sensor array 104a enables detection of whether the sensor array 104a is properly implanted within desirable tissue. Additionally, monitoring the sensor elements for proper hydration can be used as a trigger to enable at least one of determining or detecting a physiological state, data recording, and/or data transmission.

Electrochemical impedance spectroscopy (EIS) applied across any electrode pair within the sensor array 104a can be used to measure or infer tissue impedance to determine tissue hydrations levels, or a fluid status within subcutaneous tissue of a subject being monitored. Utilizing the sensor array enables continuous monitoring of tissue impedance to detect changes in fluid content within the interstitial space. In some embodiments, an EIS scan across specific frequencies is used to correlate impedance with sodium concentrations within tissue surrounding the sensor via either a lookup table or via an equivalent circuit model. Regardless of how impedance is determined, real-time monitoring can enable EIS measurements over time to determine if there is an increase in fluid within interstitial fluid based on changes in salinity of the interstitial fluid. If salinity decreases, one can infer there is additional fluid build up. Conversely, if salinity increases, it can be inferred that the fluid level within the interstitial fluid is decreasing. Increased, or increasing fluid within interstitial fluid results in lower relative impedance, measurable across multiple frequencies.

Rapid changes in tissue impedance may be correlated with changes in hydration which can be correlated to detecting a physiological state such as, but not limited to a sleep state, an exercise state, an exercise state (e.g., strenuous exercise), and a meal intake state. In some embodiments, fluid status or hydration of a subject contributes to detecting a physiological state because fluid status provides context and a normalizing factor for other measurements, such as, but not limited to tissue oxygen levels and concentrations of ROS. Absolute and trend information derived from tissue hydration levels enable adjustment or modifications to detecting a physiological state. In some embodiments, tissue hydration levels enable additional insight regarding perfusion of analytes within different types of tissues. For example, in various embodiments tissue hydration levels for a sensor array 104a placed in muscle provides additional or less information than a sensor array 104a that is placed in adipose tissue.

In some embodiments, data from a single analyte sensor (e.g., glucose) is combined with data from any one to all of the physical characteristic sensors 110 to help detect a physiological state. In other embodiments multiple analytes measured from a single probe 104 are used in conjunction with the data from any one to all of the physical characteristic sensors 110 to detect a physiological state. The rationale for detecting physiological states based on less than every data stream from the sensor array 104a is to enable tailoring of the sensor array 104a to a particular environment. For example, if a patient is breathing with assistance of a ventilator or receiving oxygen via an oxygen mask, it may not be preferred to have a risk score factor in data acquired via the tissue oxygen sensor and ROS. Alternatively, when using an ventilator or using an oxygen mask it may be advantageous to include tissue oxygen and ROS in order to determine efficacy of therapy via expected or predicted changes in the microcirculation.

With continued reference to FIG. 1, detecting a physiological state can be accomplished by analyzing real-time data from the sensor system 102 for trends in the data that are indicative of a physiological state. In some embodiments, detecting a physiological state is based on data from the sensor array 104a exceeding a threshold value. In some embodiments, the threshold values for various physiological states are associated with data from only the analyte sensors 104. In other embodiments, the threshold values for some physiological states are associated with data from the analyte sensors 104 and/or the physical sensors 110. In still other embodiments, threshold values for physiological states are set based on data from the physical sensors 110. Threshold values can be associated with absolute changes or rates of change of a single analyte, multiple analytes with or without additional absolute changes or rates of change data from the physical sensors 110. In still other embodiments, detecting a physiological state is based on a change in data from the sensor array relative to historical data from the sensor array. Typical changes in data that may be detected include, but are not limited to, changes in value, rate, coefficient of variance, and the like.

Once a threshold value for a physiological state has been crossed, a probability of the physiological state can be determined. As additional data is acquired from the sensor array the probability of the physiological state is updated. Operating as a standalone continuous glucose monitoring system data associated with detection of a physiological state can be saved for later review in order to refine the detection algorithm. When used in conjunction with an artificial pancreas system the detection of a physiological state can be used to automatically adjust basal and bolus insulin delivery.

In many embodiments, the analyte sensor system 102 is intended to be placed in subcutaneous tissue where the plurality of working electrodes within the sensor array 104a produce signals related to the analyte each transduced is configured to measure or detect (e.g., glucose, tissue oxygen, lactate, ROS, ketones). In embodiments where the sensor probe 104 is intended to be placed within subcutaneous tissue, the analyte sensor probe 104 may also be referred to as a probe. Placement within subcutaneous tissue enables a unique perspective for an oxygen sensor that is substantially different from common SpO2 oxygen measurements. Specifically, with embodiments of the analyte sensors 104, oxygen within tissue is being measured rather than a measurement of SpO2 that is an estimation of arterial oxygen. When detecting a physiological state it is advantageous to measure oxygen within tissue rather than estimated arterial oxygen because oxygen within tissue is a direct measurement of oxygen perfusion. Tissue oxygen provides insight into exercise as tissue oxygen levels tend to decline with exercise. Identification of exercise via changes in tissue oxygen levels can automatically reduce, prevent or delay basal insulin delivery that could create a risk of serious hypoglycemia. Tissue oxygen can further provide insight to meal ingestion as there have been measurable increases in muscle oxygen consumption within 15 minutes after a meal. Changes in tissue oxygen further provide insight into when a subject sleeps, with some data indicating changes in oxygen concentrations offer more reliable detection of sleep than accelerometers.

In many embodiments the analyte sensors 102 include transducers configured to measure ROS. In some embodiments, a two-electrode system is employed where each of the working electrodes electrochemically measure a particular analyte relative to a counter electrode. In other embodiments, a three-electrode system is employed where each of the working electrodes electrochemically measure a particular analyte relative to a counter and reference electrode. In one embodiment, ROS would be enabled via a pair of electrodes. An ROS measurement can be acquired through a first pair of electrodes that includes a standard working electrode and a combined counter/reference electrode, or pseudo-reference electrode.

In preferred embodiments the ROS electrodes would provide calibration free, real-time concentration levels of oxidizing agents. In many embodiments ROS measurements may provide some insight into oxidative stress. It has been shown that ROS increases at times of stress and illness, both of which impact glucose and insulin dynamics. Additionally, oxidative stress has been demonstrated to impact the timing of postprandial glucose excursions, causing a delay in glucose decline after a meal. Because of the involvement of oxidative stress in many diabetes complications, concentrations of ROS can also provide information on the risk of developing secondary diseases.

In still other embodiments, the analyte sensors 104 include transducers configured to measure lactate. Lactate can become elevated with serious illness and can further be used to help differentiate between anaerobic and aerobic activity. Users partaking in high-intensity interval exercise experienced a greater increase in glucose. The increase corresponded with a greater increase in lactate compared to those participating in moderate-intensity exercise. Accordingly, supplementing glucose data with lactate data can provide insight into intensity of exercise that can be used to predict post-exercise behavior of glucose and corresponding insulin requirements.

In some embodiments, each working electrode has a corresponding counter electrode while in other embodiments multiple working electrodes share a counter electrode. In still other embodiments, two working electrodes share a counter electrode while the third working electrode has a dedicated discrete counter electrode. Furthermore, the various embodiments of working electrodes and counter electrodes can be distributed among separate and discrete substrates. Typically, working electrodes and counter/reference electrodes are formed on a single substrate. However, an electrode design intended for use in the invention allows the complete physical separation of any of the working electrodes and any of the counter/reference electrodes. For example, as is shown in FIGS. 4A and 4B working electrodes for analyte sensors can be formed on A-side 300a while counter/reference electrodes are formed on B-side 300b. While the various electrodes may be separated on distinct A-side 300a and B-side 300b, in many embodiments the sensor array 104a having the plurality of working electrodes is inserted into the subcutaneous tissue via a single point of insertion. The use of a single insertion point minimizes both patient discomfort associated with insertion and insertion complexity.

Exemplary transducer structures that can be used for the analyte sensors 104 can be found in the following U.S. patent applications: Ser. No. 15/472,194 filed on Mar. 28, 2017; Ser. No. 16/054,649, filed on Aug. 3, 2018; Ser. No. 16/152,727 filed on Oct. 5, 2018; each of which are hereby incorporated by reference for all purposes. Additionally transducer structures can be found in PCT application serial no. PCT/US18/38984 filed on Jun. 22, 2018, which is hereby incorporated by reference for all purposes.

In many embodiments, the preferred tissue to insert the probe containing the analyte sensors 104 is subcutaneous tissue. However this should not preclude the use of the probe in other tissues such as, but not limited to skeletal muscle tissue, smooth muscle tissue or even organ tissue. Insertion of an oxygen sensor into any of these types of tissues can provide insight into the microcirculation of the specific tissue, and accordingly, the relative health of the subject.

The specific embodiments described above regarding the analyte sensors 104 and the physical sensors 110 should not be construed as limiting. In other embodiments the number of analyte sensors that can be placed on a single probe is only limited by the physical size of the probe and the willingness of subjects to insert the probe. That is to say, it should be understood that a single probe measuring glucose and any number of additional analytes should be construed as being within the current disclosure. In order to accommodate additional analyte sensing electrodes it may be necessary for some analytes to share a common reference electrode. Furthermore, regarding reference electrodes, while FIG. 4B includes multiple pseudo-reference electrodes, various other embodiments can use various combinations of pseudo-reference electrodes, discrete counter electrodes, discrete reference electrodes and various combinations thereof.

FIG. 4C is an exemplary cross-section illustration of the analyte sensor 300a illustrating diffusion of analyte, reactant and reaction by-product within one working electrode in accordance with embodiments of the present invention; any sensor probe of the present disclosure may be configured to operate according to similar analyte, reactant, and/or reaction by-product diffusion as shown in FIG. 4C. The embodiment illustrated in FIG. 4C is based on the use of glucose oxidase as a first reactive chemistry 440, thereby enabling the electrode to generate hydrogen peroxide that correlates to the concentration of glucose based on the chemical reaction represented in equation (1) below:

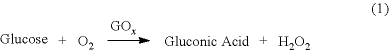

Upon insertion of the sensor assembly into a subject concentration of analytes (glucose in this embodiment) and other biomarkers around the sensor will be higher than within the individual electrodes of the sensor assembly. Concentrations of analytes and biomarkers will attempt to achieve equilibrium within the first transport materials 438 of the sensor resulting in glucose from the fluid surrounding the sensor assembly being drawn into the first transport layer 438. Fluid surrounding the sensor assembly can enter the sensor via the hydrophilic first transport material 438 but fluid cannot enter through the hydrophobic second transport material 442. In FIG. 4C glucose, represented as a ‘G’ within a hexagon, is shown entering the first transport material 438. Furthermore, oxygen, represented as ‘O₂’ is shown being supplied from the second transport materials 442 to the first reactive chemistry 440. The glucose and oxygen react with the first reactive chemistry 440 according to the chemical reaction described above resulting in the creation of by-products gluconic acid and hydrogen peroxide. The by-product hydrogen peroxide, shown as ‘H₂O₂’ in FIG. 4C, is transported via the first transport material 438 to the electrode reactive surface 416 of an electrode 436, a window to which is defined by an insulator layer 432 (e.g., masked insulator), where an applied electrical potential reduces it based on the reaction represented in equation (2) below:

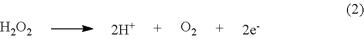

where the 2e⁻ is the electrical current picked up by the counter electrode. The consumption of glucose within the electrode lowers the concentration of glucose within the first transport layer 438 establishing a diffusion gradient that strives to reach equilibrium by bringing in additional glucose from the fluid surrounding the sensor assembly.

When compared to some glucose sensors that utilize a glucose limiting membrane (GLM), the electrode illustrated in FIG. 4C is easily identifiable as different in that the first reactive chemistry 440 is physically separated from the electrode reactive surface 416 by the first transport materials 438. The physical separation of the first reactive chemistry 440 from the electrode reactive surface 416 requires specific selection of the first transport material to support fundamental changes to diffusion within the electrode. For example, the first transport material 438 must support diffusion of the desired analyte in addition to the by-product of the analyte and the first reactive chemistry. Furthermore, in many embodiments it is desirable that the first transport material 438 also enables diffusion of the by-products of the electrochemical reaction occurring at the electrode reactive surface 416.

Placement of the first transport material 438 over the opening 444, and subsequent placement of the first reactive chemistry 440 over the first transport layer 438 moves the enzymatic reaction between analyte and first reactive chemistry 440 away from the electrode reactive surface 416. The separation of the enzymatic reaction and the electrochemical reaction reduces or minimizes the likelihood of localized pH fluctuations that accompany the electrochemical reaction that can have a negative impact on the first reactive chemistry 440. An additional benefit of the floating electrode is the first transport material 438 pathway that extends completely under the first reactive chemistry 440 that enables laterally diffusing analyte to be transported under and across the longest surface of the first reactive chemistry 440. After the enzymatic reaction, the by-product of the enzymatic reaction is consumed by the electrochemical reaction occurring on the electrode reactive surface 416. Accordingly, with hydrogen peroxide producing enzymatic reactions, the first transport material 438 pathway separating the electrode reactive surface 416 and the first reactive chemistry 440 enables analyte and by-products of the enzymatic reaction to move in substantially opposite directions.

An additional benefit of placing the first reactive chemistry 440 between the first 438 and second 442 transport materials is improved manufacturability. In many embodiments, the first reactive chemistry 440 is a mixture, blend or suspension of a specific enzyme, or biorecognition molecule, within a second material such as, but not limited to, the first transport material. Thus, applying the first reactive chemistry 440 over a layer of the first transport material 438 improves manufacturability because like materials are being placed on like materials.

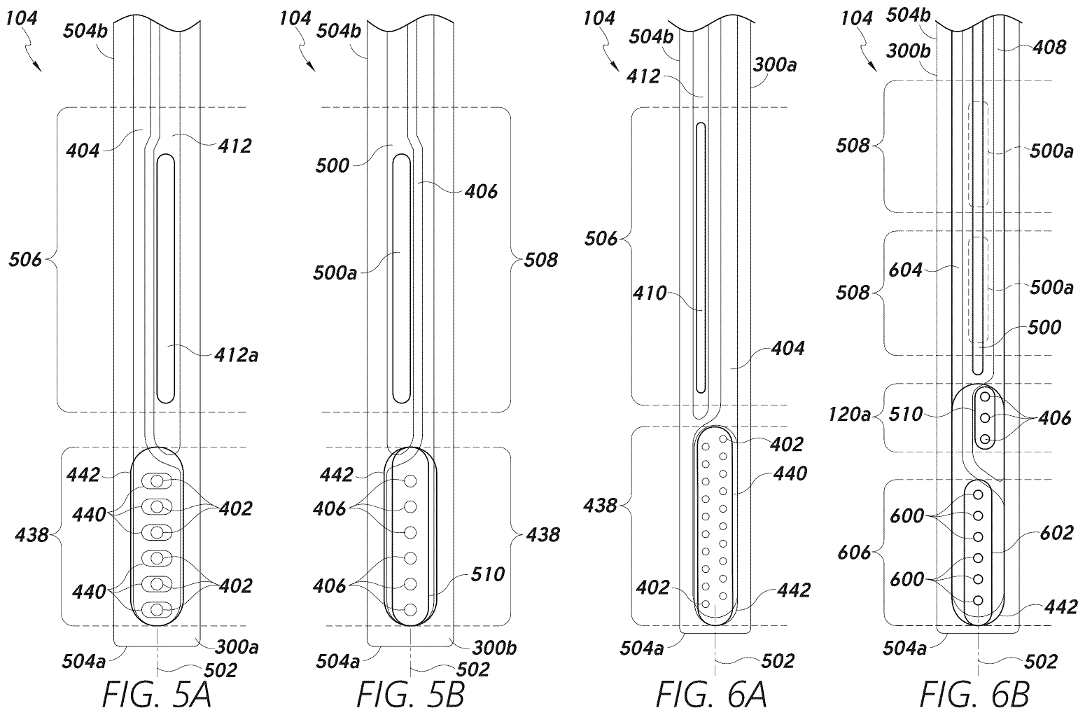

FIGS. 5A and 5B are an exemplary illustration of A-side 300a and B-side 300b of a sensor probe 104, in accordance with other embodiments of the present invention. FIG. 5A is an illustration of A-side 300a of the sensor probe 104 where the third electrode trace 412 supports a first counter/reference electrode. FIG. 5B is an illustration of B-side 300b of the sensor probe 104 where a fourth electrode trace 500 supports a second counter/reference electrode. In this embodiment, the A-side 300a includes the third electrode trace 412 that further includes third electrode opening 412a located slightly offset from the centerline 502 toward the distal end 504a. A fourth transport material 506 is applied over both the third electrode opening 412a and at least a portion of the third electrode trace 412.

On the B-side 300b is fourth electrode trace 500 that further includes fourth electrode opening 500a that is offset from the centerline 502 toward the proximal end 504b. A fifth transport material 508 is applied over both the fourth electrode opening 500a and at least a portion of the fourth electrode trace 500.

FIG. 5A, an exemplary illustration of A-side 300a of the sensor probe 104 where first electrode trace 404 supports first working electrodes 402. In this embodiment, the B-side 300b includes the second electrode trace 408 that further includes second electrode openings 406 located substantially on the centerline 502 of the distal end 504a. The first reactive chemistry 440 is applied discretely over each of the first electrode openings 402. Additionally, the first transport material 438 is applied over at least a portion of the first electrode trace 404 and the first electrode openings 402.

On the B-side 300b is the second electrode trace 408 that further includes the second electrode openings 406 that are formed substantially along the centerline 502 toward the distal end 504a. A second reactive chemistry 510 is applied substantially coincident over the second electrode opening 406. Additionally, a first transport material 438 is applied over at least a portion of the second electrode trace 408 and the second electrode openings. Second transport material 442 is placed over at least a portion of the first and second electrode trace 404 and 408, the first electrode openings 402, the second electrode opening 406, the first and second reactive chemistries 440 and 510 and the first transport material 438.

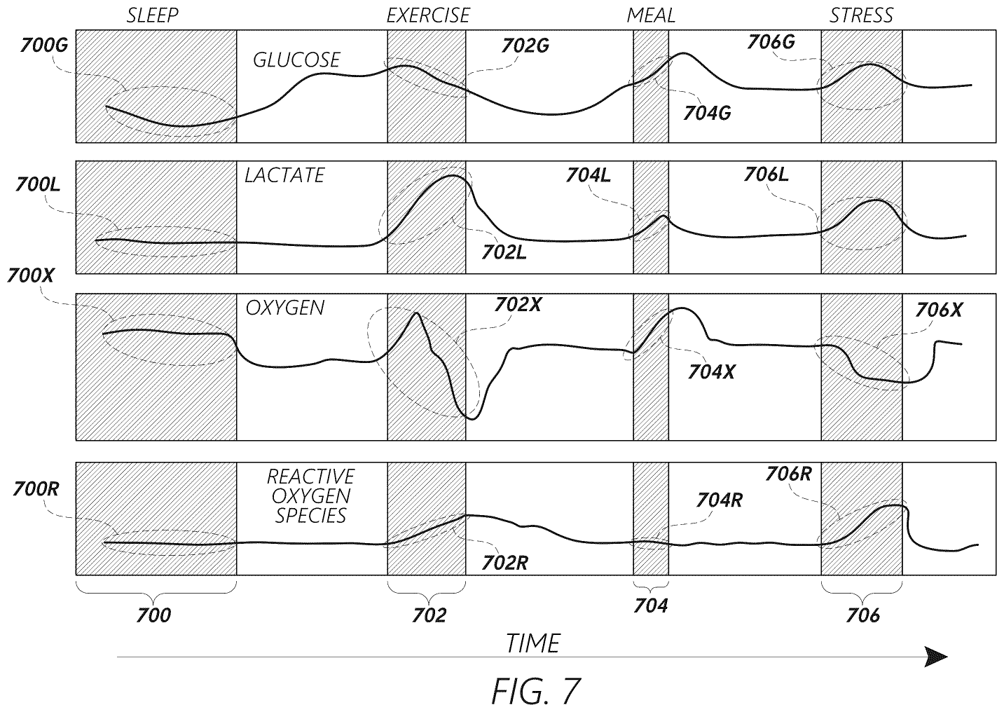

FIGS. 6A and 6B are an exemplary illustration of A-side 300a and B-side 300b of a sensor probe 104, in accordance with another embodiment of the present invention. FIG. 6A is an illustration of A-side 300a of the sensor probe 104 where the first electrode trace 404 supports a first working electrode and the third electrode trace 412 supports a first counter/reference electrode. In this embodiment, the A-side 300a includes the first electrode trace 404 that further includes first electrode openings 402 toward the distal end 504a and are also offset from the centerline 502. The first reactive chemistry 440 is applied contiguously over all of the first electrode openings 402 and at least a portion of the first electrode trace 404. Additionally, the first transport material 438 is applied over at least a portion of the first electrode trace 404 and the first electrode openings 402.

Co-located on the A-side 300a is the third electrode trace 412 that further includes third electrode opening 410 located offset from the centerline 502 toward the proximal end 504b. The fourth transport material 506 is applied over both the third electrode opening 410 and at least a portion of the third electrode trace 412.

FIG. 6B is an illustration of B-side 300b of the sensor probe 104 where second electrode trace 408 supports a second working electrode, the fourth electrode trace 500 supports a second counter/reference electrode and a fifth electrode trace 604 supports a third working electrode 410. In this embodiment, the B-side 300b includes the second electrode trace 408 that further includes second electrode openings 406 that are offset from the centerline 502. The second reactive chemistry 510 is applied contiguously over each of the second electrode openings 406 and at least a portion of the second electrode conductor 408. Additionally, the second transport material 442 is applied over at least a portion of the second electrode trace 408 and the second electrode openings 406.

Co-located on the B-side 300b is the fourth electrode trace 500 that further includes the fourth electrode opening 500a that is substantially located on the centerline 502. Note that in this embodiment, the electrode openings 500a open over a height that is greater than the height of the fourth electrode trace 500. The fifth transport material 508 is applied over both the fourth electrode openings 500a and at least a portion of the fourth electrode trace 500. Also co-located on the B-side 300b is the fifth electrode trace 604 that further includes fifth electrode openings 600 that are located substantially along centerline 502 toward the distal end 504a. A third reactive chemistry 602 is applied contiguously over each of the fifth electrode openings 600 and at least a portion of the fifth electrode trace 604.

In many embodiments, additional features or elements can be included, added or substituted for some or all of the exemplary features described above. An exemplary, non-limiting example is the use of a three electrode system (working, counter and reference electrodes) where a two electrode system (working and combined counter/reference electrodes) are discussed above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

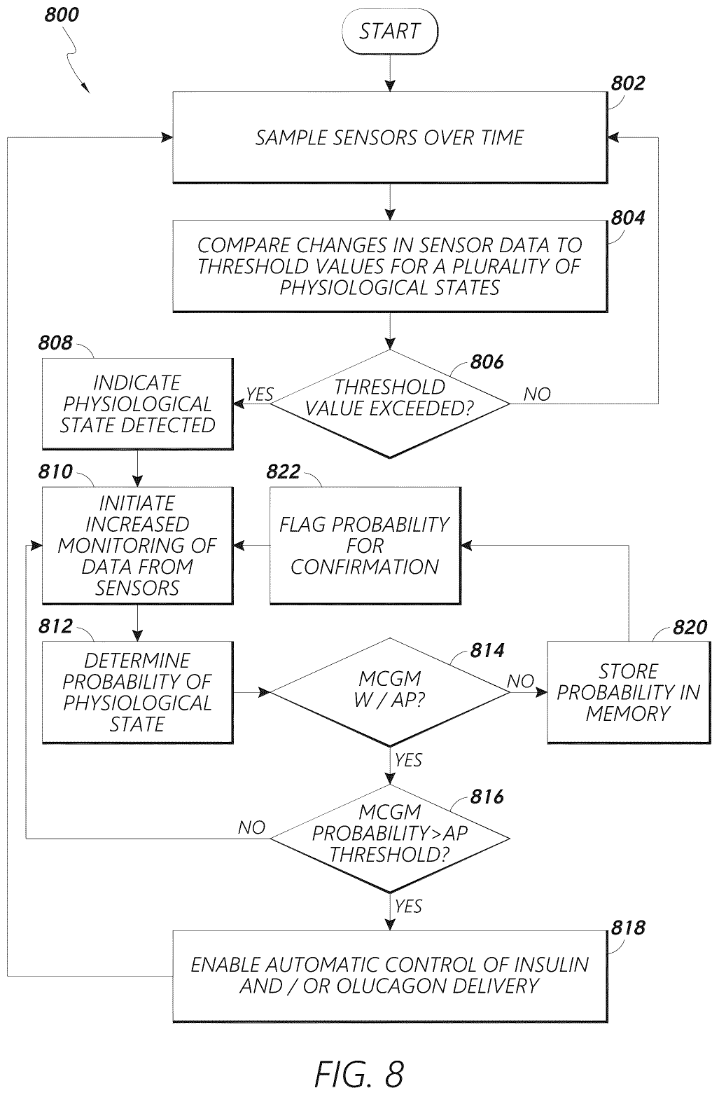

FIG. 7 is an exemplary illustration of real-time glucose, lactate, oxygen, and ROS data for various physiological states such as sleep 700, exercise 702, a meal 704 and stress 706, in accordance with embodiments of the present invention. Note that the durations of the various physiological states are not intended to be to scale. Rather the physiological states and the exemplary data/curves within the duration ranges of the physiological conditions are intended to illustrate exemplary trends and rates of change. In practice, it may be necessary to calibrate detection algorithms using days, weeks, or even months of individual patient data. Note that though the detected physiological states are shown as independent episodes, physiological states are not necessarily exclusive in time—it is possible that multiple physiological states may be detected simultaneously.

Aspects of the present disclosure provide solutions for using a sensor to measure glucose and at least one other analyte such as lactate. The glucose and lactate data acquired by the sensor, combined with additional data can enable the determination of secondary conditions. For example, a secondary condition determined from the sensor data can be an insulin condition. In some embodiments, the insulin condition is representative of insulin resistance of the subject. In other embodiments, the insulin condition may be representative of plasma insulin levels of the subject. Irrespective of the insulin condition representing insulin resistance of plasma insulin, the secondary consideration can be used as an input to define a metabolic health score for the subject.

In addition to determining individualized or personalized metabolic health scores, systems of the present disclosure can further enable refinement of personalized treatment or therapy using an insulin pump, multiple daily injections or other non-insulin pump based therapies associated with metabolic analytes such as glucose and lactate. In many embodiments, multi-analyte sensor data can be used to refine artificial pancreas or automatic insulin delivery system control algorithms. Specifically, monitoring continuous data from glucose and the second analyte like lactate can provide insights to secondary conditions such as insulin conditions and insulin dynamics that enable changes to automated insulin delivery in response to, or in anticipation of, glucose changes and/or lactate changes.

In embodiments where secondary conditions are being determined, it may be beneficial to apply a correction factor when changes in lactate or glucose levels are the result of specific activities or known situations. For example, lactate levels can be influenced when a subject is physically active. Accordingly, being able to apply a correction factor to data acquired when a subject is exercising or is generally physically active may be highly desirable to ensure secondary conditions are actually representative of physiological conditions within the subject. In some embodiments, the correction factor is applied directly to lactate data. In other embodiments, the glucose and lactate data is transformed to an initial secondary condition before a compensation or adjustment is made resulting in a corrected secondary condition. Similar to exercise, certain medications, such as, but not limited to metformin, are known to influence or change lactate levels. Accordingly, knowing if a subject is taking particular medications that influence lactate means a correction factor or transform adjustment can be made to either the lactate level or to the initial secondary condition.

Additional situations or conditions that can impact or influence lactate levels within a subject include, but are not limited to chronic disease states. Exemplary chronic disease states such as cancer, high blood pressure, type 1 or type 2 diabetes, NAFLD or COPD can all impact lactate levels within a subject. In embodiments where chronic disease states influence lactate levels, it may be preferable to apply a correction factor to all lactate levels or all initially determined secondary conditions.

As the secondary condition may be dependent on both lactate and glucose, activities, situations or conditions that influence or modify glucose levels within a subject may also benefit from the application of a correction factor or adjustment. Known events or situations that impact glucose levels within a subject include, but are not limited to consuming carbohydrates, stress, and special diets such as the ketogenic diet and intermittent fasting. When applying transform adjustments to glucose data, meals or consuming carbohydrates may be viewed as a discrete event. Accordingly, a correction factor may only be applied to a small portion of glucose data associated with a postprandial window of time. Similarly, the use of correction factors for situations like stress or special diets may be viewed as being similar to chronic conditions

In some embodiments, combinations of analyte sensor data (e.g., glucose and lactate) are used to initially detect a physiological state. In other embodiments, physiological states are initially detected using a combination of analyte sensor data and physical sensor data such as, but not limited to accelerometers, temperature sensors, hydration sensors, timers, ECG and the like. In these embodiments the physical sensors are installed or enabled via hardware and/or software within the electronics package associated with the analyte sensors. In still other embodiments, initial detection of a physiological state is determined based on physical sensor data. Regardless of how initial detection of the physiological state is determined, subsequently acquired data from either analyte sensors or physical sensors can be used to confirm or reject the initial detection.

In one embodiment glucose and tissue oxygen represent analytes being monitored. Accordingly, in FIG. 7, sleep glucose values 700G and sleep lactate 700L are actively being acquired starting from the left to the right. Initially 700G is in a shallow decline that eventually levels out with even a slight increase toward the end of the sleep state. Similarly, sleep lactate 700L is illustrated as a relatively flat line. Accordingly, in some embodiments prolonged periods of steady lactate data in conjunction with decreasing to leveling glucose data can result in an initial detection of a sleep state. In embodiments where analyte sensors are supplemented by physical sensors, exemplary physical data acquired by accelerometers, thermometers, elapsed time, and ECG systems can confirm or refute the detection of a sleep state by the analyte sensor data. In preferred embodiments, analyte sensor data and physical sensor data are examined simultaneously for an initial detection of a sleep state and continued analysis of analyte and sensor data is used to confirm or refute the initial detection. In standalone MCGM applications detection and confirmation of the detection can be written into memory for subsequent review by the patient or their physician. In AP applications, confirmation of detection can be determined by calculating a confidence score of physiological state. An AP application can be programmed to automatically perform modifications to basal or bolus therapy based on physiological states having a confidence score meeting or exceeding a predetermined threshold value.

Real-time glucose and lactate data can be utilized by systems of the present disclosure to automatically detect/determine a second physiological state, such as exercise 702. Exercise glucose 702G and exercise lactate 702L are exemplary and are not intended to be indicative of any particular individual or group of individuals. What is to be noted for each analyte is the combination of absolute change and rate of change. As exercise is performed, glucose levels may initially rise and then generally decrease. Similarly, during aerobic exercise, lactate levels may gradually increase until the exertion becomes anaerobic, where lactate levels may increase at a more rapid rate.

Meal consumption can also be identified by examining real-time glucose and lactate data. Meal glucose 704G and meal lactate 704L each show increases during, or shortly after, a meal. It may be beneficial to supplement analyte sensor data with time when attempting to detect a meal state. Specifically, it may be very beneficial to include historical meal times within an algorithm to assist in distinguishing between a meal state and an exercise state. While the meal glucose 704G data and meal lactate 704L data is exemplary, rates of change and absolute value changes of each analyte across various meals and roughly the same time can help detect a meal state with increasing confidence.

Stress is another physiological state that may be detected/determined by systems of the present disclosure, using a combination of analyte and physical sensors. Stress glucose 706G and stress lactate 706L are exemplary illustrations of data obtained during a period of stress. In the exemplary data both glucose and lactate increase and decrease a relatively modest amount. Again, the exemplary data can be supplemented by physical sensor data that can be used to increase confidence in detecting stress.

The simplest embodiments combine glucose with another analyte such as a lactate, discussed above, or oxygen or ROS. In some embodiments, glucose and two additional analytes are measured, while in still other embodiments, glucose and three other analytes are measured. Factors such as the physical size of the sensor, transducer size, and capabilities of the electronics package limit the number of analytes that can be concurrently measured with glucose.

FIG. 7 includes exemplary illustrations of sleep oxygen 700X, exercise oxygen 702X, meal oxygen 704X and stress oxygen 706X, which can be used in conjunction with glucose or other analytes to help detect the various physiological states. Oxygen changes in response to exercise and meals and can enable more reliable detection of sleep than accelerometers. Fortunately, because accelerometers can be included as physical sensors, in some embodiments, sleep oxygen 700X and be detected via either analyte or physical sensors and further confirmed using either or both analyte or physical sensors.

FIG. 7 further includes exemplary illustrations of sleep ROS 700R, exercise ROS 702R, meal ROS 704R and stress ROS 706R. Concentration of ROS in the body provides insight into oxidative stress. Additionally, oxidative stress can impact the timing of postprandial glucose excursions, causing a delay in glucose decrease after a meal. Because of the involvement of oxidative stress in many diabetes related complications, concentration of ROS can also provide information on the risk of developing secondary diseases.

The analytes illustrated and discussed regarding FIG. 7 are exemplary and should not be construed as limiting. In other embodiments additional analytes associated with diabetes, diabetes related complications, and secondary diseases may be monitored in conjunction with glucose. In some embodiments, glucose and multiple analytes are monitored to detect a physiological state. In many of those embodiments every analyte is measured while the sensor is implanted. In other embodiments, select analytes are measured only after a physiological condition is detected or another threshold triggers measurement of the select analyte. The rationale for selectively measuring specific analytes can be one of saving power, reducing potential crosstalk among the various working electrodes, reducing computational requirements, controlling memory usage and the like.

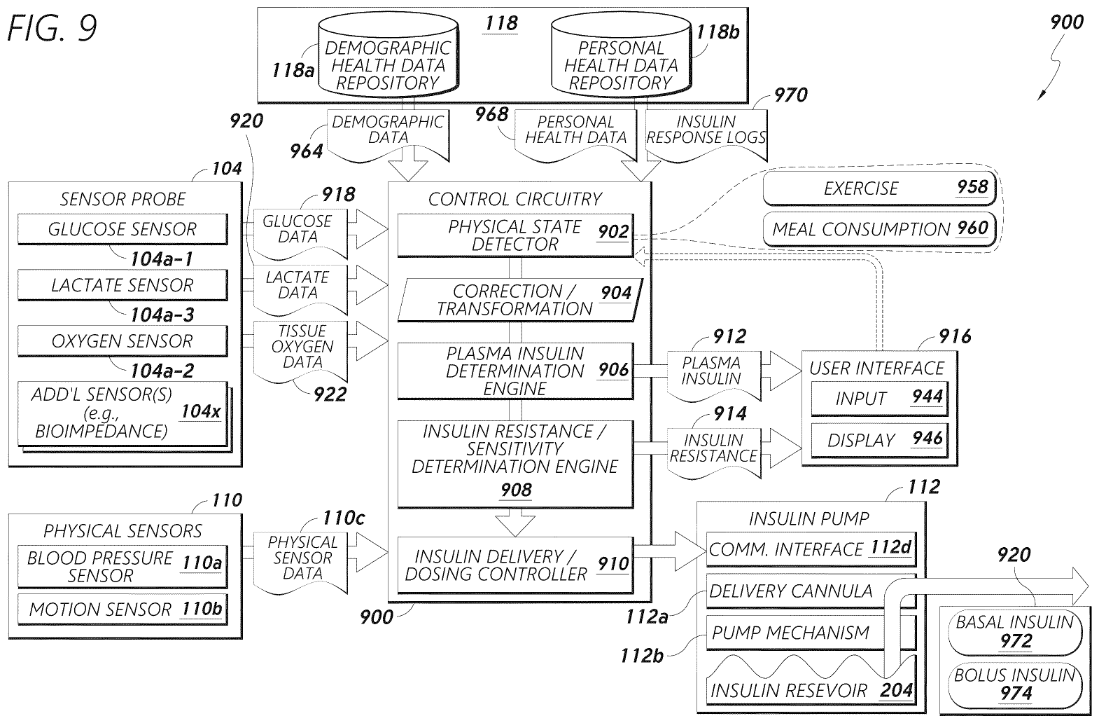

FIG. 8 is an exemplary flowchart illustrating a process 800 of detecting a physiological condition, in accordance with various embodiments of the present invention. The flowchart begins with START operation that could be viewed as being roughly equivalent to having inserted, powered, and properly hydrated, or run-in, the analyte sensor. Additionally, in some embodiments, physical sensors may be similarly ready to produce meaningful data. Operation 802 can involve sampling and storing data from both the analyte sensors and physical sensors. Operation 804 may further involve comparing changes in data streams from the sensors to threshold values for a plurality of physiological states. For example, at operation 804, data streams from the analyte and physical sensors may be processed to determine combinations of absolute changes or rates of change that exceed a threshold value indicative of a known physiological state such as, but not limited to, sleep, eating a meal, exercise or stress.

Operation 806 involves comparing whether predetermined threshold value(s) indicative of a physiological state have been exceeded. If no threshold values have been exceeded, the process 800 may return to operation 802. If a threshold value has been exceeded, the process 800 may proceed to operation 808, which may involve identifying which physiological state has been detected. Operation 810 may involve initiating increased monitoring of data from the sensors. With a physiological state detected, the system can begin interrogating analyte and physical sensors to obtain data to either confirm or refute the initial detection.

Operation 812 can involve determining or calculating a probability of the physiological state based on the increased monitoring from the sensors. Operation 814 can involve comparing whether the sensor data is associated with an artificial pancreas system. If no artificial pancreas system is associated, operation 820 stores the probability in memory while subsequent operation 822 flags the probability data for confirmation. In many embodiments, confirmed data can be processed via machine learning or predictive analytics tools to create or modify individual or multi-patient models that can be subsequently used to detect physiological states. If an artificial pancreas system is associated, the process 800 may proceed to operation 816, which may involve comparing the probability of a physiological condition to a minimum probability threshold for the artificial pancreas system. If the probability is greater than a minimum probability threshold operation 818, the process 800 may proceed to operation/block 818, which may involve enabling automatic control of insulin or glucagon delivery from the artificial pancreas system. In many embodiments, detection of a physiological state alone does not necessarily require augmentation of insulin or glucagon delivery. Rather, the detected physiological state becomes another variable in determining whether modifications to insulin or glucagon may be necessary to control a patient's glucose. If the probability of a physiological state does not meet the artificial pancreas minimum requirement the flowchart returns to either operation 802 or 810.

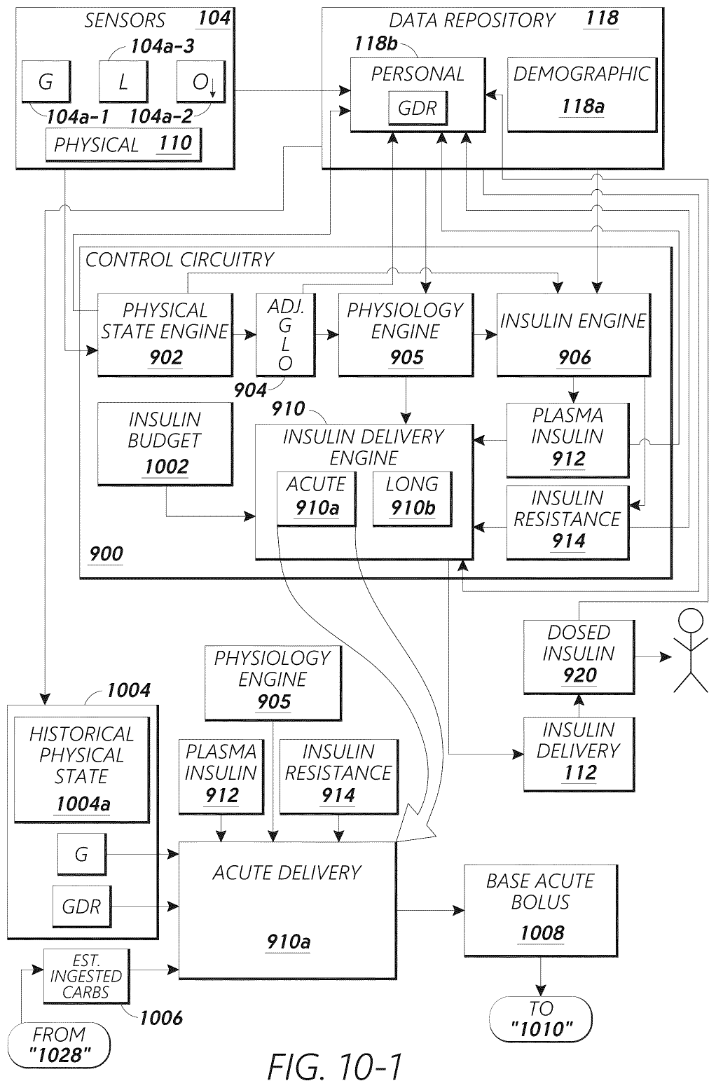

FIG. 9 is an exemplary block diagram of a system 900 for enabling insulin delivery, in accordance with embodiments of the present invention. The system 900 includes certain control circuitry 901 configured to receive input from an analyte sensor probe 104 and/or one or more physical sensors 110. The analyte sensor probe 104 is configured to provide two or more of glucose data 104b-1, lactate data 104b-3, and tissue oxygen data 104b-2 to the control circuitry 901. Exemplary physical sensor data 110c can include motion data from accelerometers 110b, tissue hydration, blood pressure, and the like. Note that in some embodiments, the physical sensor 110 may be integrated within a sensor system, such as a sensor system like the sensor system 102 of FIG. 1. However, as illustrated in FIG. 9, the physical sensor(s) 110 may be embodied in a separate system, such as a smart phone, smart watch, smart ring or the like. In still other embodiments, some physical sensors 110 are integrated with the sensor system 102 while other physical sensors are located on a third party device. The control circuitry 901 advantageously processes the raw data from the analyte sensor probe 104 and physical sensor(s) 110, to align timing and contextualize metabolic states.

The integrated analyte control system 900 is designed for real-time monitoring and management of a user's physiological state through multi-analyte sensing and responsive medication delivery. The sensor probe 104 comprises multiple analyte sensors, including a glucose sensor 104A-1 that provides glucose data 918, a lactate sensor 104A-3 that provides lactate data 920, and/or an oxygen sensor 104A-2 that provides tissue oxygen data 922. The sensor probe 104 may also incorporate additional sensor(s), such as a bioimpedance sensor 104x for measuring tissue conductivity and hydration, a reactive oxygen species (ROS) sensor to assess oxidative stress, and/or the like. These sensors can advantageously enable comprehensive analysis of a user's metabolic state, supporting tailored therapeutic interventions. The sensor probe 104 may be constructed as a flexible microelectrode array integrated with specific enzymatic coatings for target analytes, as described in detail above.

Integrating multiple analyte sensors into a single probe implanted in the tissue at the same location offers significant technological improvements and practical advantages, addressing challenges in real-time metabolic monitoring and therapeutic decision-making. By co-locating the sensors within a single probe, the system of FIG. 9 can achieve synchronized and contextually relevant measurements of multiple analytes, such as glucose, lactate, oxygen, and reactive oxygen species, directly from a common microenvironment in the tissue. This spatial correlation can reduce discrepancies caused by temporal or spatial variations that could arise from using separate sensors at different sites. For example, glucose levels measured alongside lactate in the same tissue location can provide immediate insights into whether glucose uptake is aerobic (low lactate) or anaerobic (high lactate), directly informing the body's metabolic state. Similarly, tissue oxygen data collected from the same site can advantageously add an additional layer of accuracy by contextualizing glucose and lactate levels in terms of oxygen availability, which can have a direct impact on insulin sensitivity and cellular metabolism.

In addition, utilization of an integrated multi-analyte sensor probe 104 for basal insulin and/or insulin resistance determination by the control circuitry 900 provides reduced-invasiveness solutions, as a single probe requires fewer insertion points compared to multiple, separate probes, reducing the risk of infection, tissue trauma, or patient discomfort. Furthermore, housing multiple sensor electrode sets at least partially within a single implant simplifies the design of the control and data processing systems, as all analyte signals can be captured, processed, and interpreted in a unified manner with reduced noise and cross-interference. The single-probe design can also improve device durability and calibration stability by ensuring consistent environmental exposure for all sensors, reducing variability due to differences in implantation depth or tissue type.

From a technical perspective, co-locating the sensors enhances the device's adaptability to physiological changes. For instance, during exercise or hypoxia, the simultaneous detection of glucose, lactate, and oxygen in the same tissue microenvironment can allow the system to more precisely distinguish between metabolic states such as insulin resistance versus normal activity-driven glucose uptake. This localized, real-time data fusion leveraged by the control circuitry 900 can improve the accuracy of therapeutic operations relating to insulin/glucagon delivery by the dosing controller 910, such as dynamic insulin or glucagon delivery adjustments, enabling a level of metabolic control that is more challenging with separate sensing devices/systems. These advancements demonstrate significant technological improvements over existing solutions in the field of multi-analyte monitoring and control.

The integration of multiple analyte sensors into a single probe implanted in the same tissue location can provide technological improvement in sensor timing synchronization, facilitating temporal alignment of all analyte measurements, potentially eliminating the need for complex software algorithms to reconcile time lags or offsets that could occur with separate sensors. This temporal alignment can be particularly advantageous for capturing the rapid dynamics of metabolic changes, such as those induced by exercise, meals, or insulin dosing, where analyte levels can fluctuate within seconds to minutes. For example, during physical activity, glucose levels may drop rapidly while lactate levels rise due to anaerobic metabolism. Simultaneously measuring these changes in real-time within the same tissue environment allows the control circuitry 900 to accurately interpret the metabolic state, distinguishing between insulin-driven glucose uptake and activity-driven lactate production. Similarly, in postprandial states, synchronized detection of glucose spikes alongside stable lactate and oxygen levels ensures precise identification of insulin secretion adequacy or insufficiency. Without this synchronization, discrepancies in timing could lead to inaccurate interpretations, such as falsely attributing delayed glucose changes to metabolic abnormalities.

With regard to the control circuitry hardware, unified timing provided by a single probe 104 for multiple analyte sensor electrodes can simplify data acquisition and processing, as signals from all sensors can be sampled through a shared interface with consistent timing intervals. This reduces the complexity and computational load on the control circuitry 900, enabling faster and more reliable data processing/transformation. Additionally, synchronized sensor data enhances the reliability of machine learning models of the system 900 (e.g., any of the modules 902, 906, 908, 910 of the control circuitry 900 can comprise trained model(s)) by providing a coherent dataset for training and decision-making.

The one or more physical sensors 110 can be configured to provide signals indicating aspects of the user's physiological state. Such sensors may include a blood pressure sensor 110a for indicating vascular health, a motion sensor 110b for tracking activity levels and exercise, and/or other physical sensors such as thermometers for body temperature monitoring. These physical sensors 110 provide additional inputs to improve the accuracy and adaptability of the system in response to changing user conditions.

Control and management of the system 900 can be performed by the control circuitry 901, which can be integrated in a single device or component or distributed across various devices/components of the system 900. The control circuitry 901 can be configured to manage data transfer and transformation, analyte monitoring, and medication delivery for insulin and/or glucagon. The control circuitry 901 can include a physical state detector 902 that is configured to determine the user's physical state based on inputs from sensors and/or user interaction via a user interface 916. The physical state detector 902 can identify states such as exercise or other motion-related activity, meal consumption, or fasting based at least in part on data from the motion sensor 110b, user inputs, and/or other contextual information. For example, the user may interact with the user interface 916, which can include a display 946 for presenting system information and a user input mechanism 944, such as a touchscreen or physical button(s), to indicate physical activities or meal intake. The physical state detector 902 is configured to analyze these inputs to determine the user's current state (e.g., exercise state 958, meal consumption state 960) and adjust the system's response accordingly.

The control circuitry 901 includes a plasma insulin determination engine 906 that is configured to calculate plasma insulin levels based on data from the glucose sensor 104A-1, lactate sensor 104A-3, and/or oxygen sensor 104A-2. The calculated plasma insulin levels 912 can be used to guide medication delivery decisions and/or are presented to the user via the user interface 916. Additionally, the control circuitry 901 can feature an insulin resistance/sensitivity determination engine 908, which evaluates the user's metabolic responsiveness to insulin based on sensor data and historical trends. Insulin resistance levels 914 are similarly displayed to the user and used as critical inputs for insulin and/or glucagon dosing decisions. The plasma insulin determination engine 906 and/or insulin resistance/sensitivity determination engine 908 can advantageously operate on and/or using transformed sensor data rather than on the raw glucose 918, lactate 920, and/or oxygen 922 data, such data transformation being implemented at least in part by the transformation engine 904.

The control circuitry 901 can generate the plasma insulin 912 based at least in part on the lactate data 920, which reflects anaerobic metabolism, which can occur during intense exercise or hypoxia, for example. Elevated lactate suggests increased glucose utilization and altered insulin sensitivity. The plasma insulin determination engine 906 can generate data and/or signals indicative of present plasma insulin levels of the user based on the physiological relationships between the glucose data 918, lactate data 920, and/or tissue oxygen data 922, as well as insulin activity. For example, glucose data indicating elevated blood glucose levels can trigger the control circuitry 901 to initiate or increase insulin delivery to facilitate glucose uptake into cells. The engine 906 can determine the endogenous plasma insulin levels of the user based on determined rates of rise and/or fall of glucose levels, possibly in conjunction with insulin administration log data. Furthermore, where the lactate data 920 indicates elevated lactate, the control circuitry 901 can determine/detect conditions of increased anaerobic metabolism due to insufficient insulin-mediated glucose uptake. For example, when cells are not effectively utilizing glucose because of low insulin levels or insulin resistance, glycolysis can increase, leading to higher lactate production. Furthermore, low tissue oxygen indicated by the tissue oxygen data 922 can be used as indicating exacerbated anaerobic metabolism, which can have an influence on lactate production and glucose utilization.

The insulin resistance/sensitivity determination engine 908 can be configured to detect high glucose levels coupled with normal lactate levels indicating insulin resistance. Where the glucose data 918 and lactate data 920 indicate both high glucose and high lactate levels, the plasma insulin determination engine 906 may determine relatively low plasma insulin levels are present, which can result in inadequate glucose uptake and increased anaerobic metabolism.

To determine insulin resistance or sensitivity, the engine 908 can monitor glucose and lactate levels over time, particularly after meals or during periods of physical activity. If glucose levels remain elevated despite normal or high plasma insulin levels as determined by the engine 906, the engine 908 may determine relatively high insulin resistance levels. Elevated lactate levels in this context can further increase insulin resistance determination by the engine 908, as such conditions may indicate cells resorting to anaerobic metabolism due to impaired glucose uptake. By contrast, if glucose levels decrease appropriately in response to insulin and lactate levels remain stable, the engine 908 may determine such data is indicating insulin sensitivity.

Medication dosing control can be managed by an insulin delivery/dosing controller engine 910, which generates precise control signals for the insulin and/or glucagon pump 112. Such signals can be based on plasma insulin levels 912, insulin resistance 914, and/or other input(s), such as historical and/or demographic data. The system 900 incorporates data from one or more data repositories, such as a health data repository 118, which stores demographic health data 118a (e.g., population-specific insulin responses or metabolic profiles) and personal health data 118b (e.g., the user's medical history, previous analyte responses, and dosing logs). For example, demographic data 964 and personal health data 968, combined with insulin response logs 970, can serve as a basis for the control circuitry 901 to adjust dosing control dynamically, ensuring personalized and effective medication delivery.

The insulin and/or glucagon pump 112 includes a communication interface 112d that receives control signals from the control circuitry 901 to regulate medication delivery through a delivery cannula 112a. Medication(s) are stored in reservoir(s) 204 within, or otherwise in fluid communication with, the pump 112. The reservoir(s) 204 can include a single reservoir for insulin or dual reservoirs for both insulin and glucagon. The pump mechanism 112b is configured to deliver basal insulin 972 (and/or glucagon) continuously and/or bolus insulin 974 as needed, with optional glucagon delivery to prevent or counteract hypoglycemia. The pump mechanism may utilize peristaltic, piezoelectric, or microfluidic actuation for precise dosing. By integrating real-time data from the sensor probe 104, contextual inputs from physical sensors 110, and/or historical data repositories 118, the system 900 dynamically adjusts medication delivery to optimize the user's metabolic health while minimizing risks such as hypoglycemia or hyperglycemia.

By combining advanced sensing, adaptive control algorithms, and personalized data analysis, the system 900 offers a comprehensive solution for managing complex metabolic states. The system 900 advantageously provides mechanisms to detect and respond to real-time physiological changes with tailored insulin and/or glucagon delivery, which represents a significant technological improvement over existing systems, demonstrating a concrete application of innovative data transformation and control processes.

In addition to detecting physiological conditions based on sensor data discussed above, in many embodiments, data from the sensor system 900 may be utilized to determine (e.g., indirectly) conditions such as, but not limited to, insulin conditions for a subject. Specifically, in preferred embodiments, sensor system data can be transformed to determine either real-time insulin sensitivity or real-time plasma insulin for a subject. In preferred embodiments, transformation of glucose and lactate data from the analyte sensor based on supplemental data from the data repository enables determination of an insulin condition. In other embodiments, there may be situations or instances where it may be advantageous to apply a correction or compensation to the insulin condition.

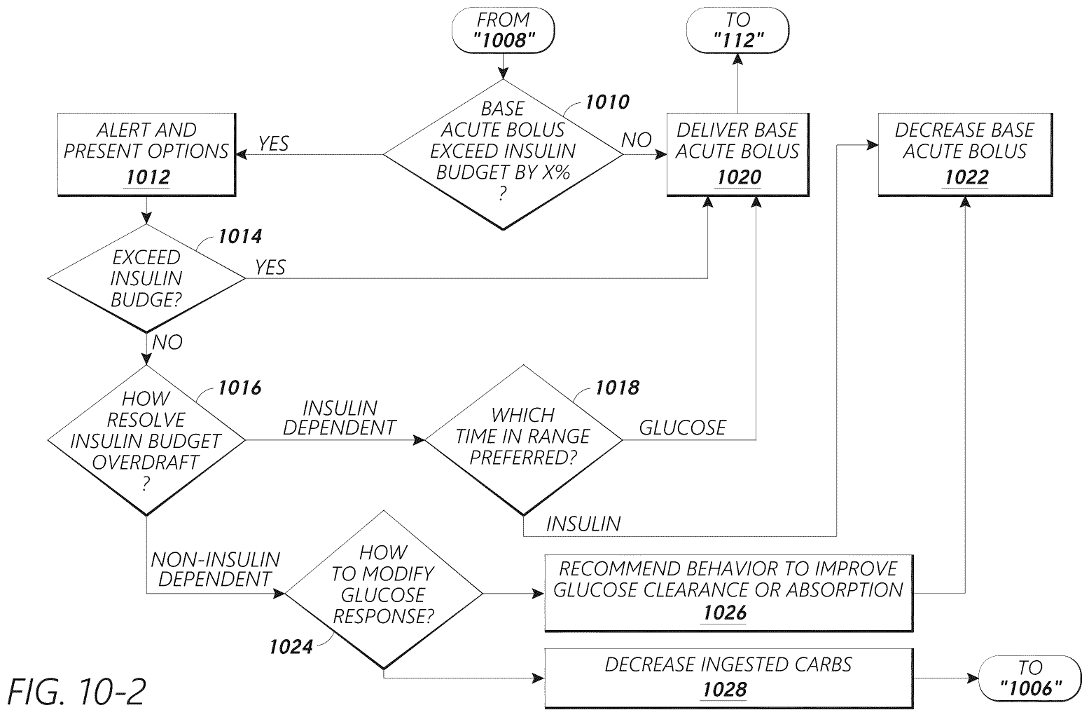

FIGS. 10-1 and 10-2 (collectively referred to as ‘FIG. 10’) show an exemplary hybrid block/flow diagram that illustrates interactions between various components within an analyte management system, such as the system 100 of FIG. 1 and/or the system 900 of FIG. 9, in order to deliver insulin in accordance with embodiments of the present invention. Additionally, FIG. 10 further illustrates operations within the insulin delivery dosing/controller engine 910 that are intended to reduce the likelihood of an exogenous insulin user developing insulin resistance. The glucose sensor 104a-1, the oxygen sensor 104a-2, the lactate sensor 104a-3, and physical sensor(s) 110 are illustrated as being part of the analyte sensor probe 104. In alternate embodiments, the physical sensors 110 can be either completely separated from the analyte sensor probe 104 or separate physical sensors 110 can supplement physical sensors 110 integrated with the analyte sensor probe 104.

Output from the sensor probe 104 (including physical sensor data) can be stored within the personal data repository 118b. The personal data repository 118b and the demographic data repository 118a are illustrated as being within the data repository 118. Control circuitry 900 includes a physical state engine 902 that is configured to output adjusted glucose, lactate and oxygen (GLO) data 904. Additionally, the adjusted GLO data 904 may be stored in the personal health repository 118b, and may also be used as an input to a physiology engine 905. An additional input to the physiology engine 905 is data from the personal data repository 118b. Data from the personal data repository 118b that can be used as an input to the physiology engine includes, but is not limited to age, LDL cholesterol, sex, chronic conditions (e.g., type 1, type 2 diabetes, heart disease), medication, and the like. The exemplary types of data discussed above should not be construed as limiting. Rather the exemplary types of data are intended to be illustrative of any type of medical record that can influence or have an impact upon metabolic health. The physiology engine 905 can be configured to determine a transform or correction factor based on the input of the adjusted GLO data 904 and/or the data provided by the data repository 118. In some embodiments, the physiology engine 905 is configured to normalize and weigh factors such as chronic conditions and medication relative to their respective impacts on metabolic health. The output of the physiology engine 905 can be used as an input to both the insulin engine 906 and the insulin delivery engine 910.

The insulin engine 906 receives data from the data repository 118, the physiology engine 905 and the physical state engine 902. The output of the insulin engine 906 is a plasma insulin value 912 and an insulin resistance value 912. In many embodiments, the insulin engine 906 uses one or more insulin models to determine the plasma insulin value 912 and the insulin resistance value 912. Exemplary insulin models that can be applied within the insulin engine 906 can be found described in Bergman et al., Dalla Man et al., and Hovorka et al. Various insulin models within the insulin engine 906 may require different inputs that can be supplied from the data repository 118. Additionally, in some embodiments input to the insulin engine 906 from the physiology engine 905 can be applied individually to each model within the insulin engine 906. In other embodiments, the physiology engine 905 is applied selectively to models within the insulin engine 906 that meet a threshold of requirements. In still other embodiments, each insulin model maintained and/or utilized by the insulin engine 906 may receive a weighting based on the completeness of inputs from the physiology engine 905.

Both the plasma insulin value(s) 912 and insulin resistance value(s) 914 that are output from the insulin engine 906 may be used as input to the insulin delivery engine 910. Additional inputs to the insulin delivery engine 910 include data from the data repository 118, output from the physiology engine 905, and an insulin budget 1002. In some embodiments, the insulin budget is a preferred quantity of insulin that is intended to be delivered to a user over a defined period of time. For example, in some embodiments the insulin budget 1002 may be a number of units of insulin per day, per week, per month and the like. The intent of the insulin budget is to provide a safeguard for the user against over-delivery of insulin that, overtime, can increase the likelihood of becoming insulin resistant. The control circuitry 900 is configured to track the insulin budget 1002, such that as insulin is delivered (e.g., bolus or basal insulin), a tracked residual of the insulin budget 1002 reflects the amount of insulin from the original budget that remains yet undelivered for the present time period.

The insulin engine 910 includes an acute engine 910a and a long term engine 910b. In many embodiments, the acute engine 910a is intended to determine a quantity of insulin to address acute glucose excursions. In many embodiments, the output of the acute engine 910a may alternatively be viewed as a bolus quantity of insulin to address glucose excursions associated with meals or exercise. Similarly, the long term engine 910b can alternatively be viewed as determining a quantity of insulin to address long term glucose effects, or more akin to basal insulin delivery.

Details associated with inputs and operations associated with the acute engine 910a are also included in FIG. 10. Inputs to the acute engine 910a can include plasma insulin 912, insulin resistance 914, the physiology engine 905 output, data from the data repository 118, and/or an estimated carbohydrate input 1006. In FIG. 10, data from the data repository 118 is illustrated as a subset 1004, that is intended to be exemplary, representative of data that can be supplied or drawn from the data repository 118. The subset 1004 includes historical physical state data 1004a, current and historical glucose data (‘G’), and the glucose disposal rate (‘GDR’) for the subject. The data within the subset 1004 should not be construed as limiting. In other embodiments, additional real-time or historical data from the data repository 118 may be used as an input to the acute engine 910a. The inclusion of historical physical state data 1004 enables the insulin delivery engine 910 to compensate for changes in metabolism associated with physical activity such as, but not limited to exercise or other sustained physical movement.

From the inputs described above, the acute engine 910a determines a base acute bolus 1008. Operation 1010 determines if the base acute bolus exceeds the insulin budget 1002 by a specified percentage, or exceeds a fractional or pro rata dose/portion (e.g., in an absolute sense or exceeds or approaches a predetermined percentage or range of the fractional/pro-rata dose/portion) that corresponds to the portion of time of the dose relative to the full time period associated with the insulin budget. In some embodiments, the specified percentage/range is established by a physician. In other embodiments, the specified percentage/range may be dynamically varied based on how much time remains in the insulin budget time period. For example, if an insulin budget is for a 24 hour period and the acute engine 910a is being engaged within the first six hours, the specified percentage over the insulin budget may be higher than if the acute engine 910a is being engaged in the last six hours of the 24 hour period.

If operation 1010 determines that the base acute bolus 1008 does not exceed the insulin budget (or fractional/pro-rata portion thereof or associated range/threshold), operation 1020 involves providing control signal(s) to the insulin delivery module 112 to direct and/or cause delivery of the base acute bolus. If operation 1010 determines that the base acute bolus 1008 does exceed the insulin budget, operation 1012 may alert the user. Operation 1014 prompts the user to confirm if the user wishes to exceed their insulin budget. If the user chooses to exceed the insulin budget, operation 1020 sends instructions to the insulin delivery module 112 to deliver the base acute bolus. If the user does not wish to exceed the insulin budget, operation 1016 prompts the user to decide how to resolve the overdraft from the insulin budget. Exemplary, non-limiting options to resolve the insulin budget overdraft include a resolution that is insulin dependent and a resolution that is non-insulin dependent.

If a user chooses to go with an insulin-dependent resolution, operation 1018 may involve prompting the user to choose whether to tailor the insulin bolus toward keeping glucose or insulin within a preferred range. In many embodiments the preferred range for both insulin and glucose may be expressed as a time-in-range. In other embodiments, the preferred range for both insulin and glucose may be established as a threshold value. In still other embodiments, one or both of the glucose or insulin preferred range may be a combination of a time-in-range and a threshold value. The exemplary embodiments of a preferred range described above are intended to be non-limiting. Other embodiments may use different metrics associated with both or either glucose and insulin that demonstrate or illustrate a level of control over excursions of either or both analytes.

The intended purpose of quantitatively demonstrating control over both glucose and insulin is to enable a user to intentionally modify delivery of exogenous insulin to either (a) sacrifice glucose control to minimize insulin delivery relative to the insulin budget or, (b) overdraft from the insulin budget to maintain better control of glucose. Use of the insulin budget enables a user to better balance glucose control and total insulin delivered over a period of time. One benefit of being able to intentionally bias exogenous insulin delivery toward glucose control or insulin delivery is the ability to balance glucose control with the danger of delivering so much insulin that a subject or user develops insulin resistance.

For example, in many examples, the preferred metric of efficacy for users of insulin pumps is the user's HbA1c. However, in many cases, the desire to drive glucose low for the preferred HbA1c results in the over-delivery of insulin. The resulting development of insulin resistance, and the associated comorbidities, can prevent insulin pump therapy users from achieving the anticipated improved health spans associated with better glucose control. An alternate perspective is to view the over-delivery of exogenous insulin as being similar to the physiological conditions that lead to the development in type 2 diabetes.

In some embodiments, if operation 1018 is resolved with a preference of having glucose be within a time-in-range, operation 1020 can deliver the base acute bolus. In alternative embodiments, operation 1018 may automatically reduce the base acute bolus by a preset percentage resulting in a reduced acute bolus (see block 1022). In other embodiments, rather than a preset percentage, the control circuitry 901 can determine a reduced acute bolus that results in a user having a glucose level that is a set percentage above the preferred level. In some embodiments, reduction of the acute bolus is associated with corresponding levels or grades of both glucose and insulin control. For example, both glucose and insulin can each have levels associated with “good,” “medium,” and “poor”. In exemplary embodiments each “good,” “medium,” and “poor” glucose level may be a target glucose value after X minutes/hours after delivery of the reduced acute bolus. An exemplary “good” glucose value may be between X and X′ mg/dL. An exemplary “medium” glucose value may be between Y and Y′ mg/dL. An exemplary “poor” glucose value may be between Z and Z′ mg/dL. Similarly, insulin may have associated good, medium and poor levels associated with, in some embodiments, percentages of insulin budget used or estimated insulin resistance after X minutes/hours after delivery of the reduced bolus. The use of the levels associated with glucose and insulin can allow a user to enter a preference for various combinations of glucose and insulin levels that enable the control circuitry to automatically determine the reduced acute bolus. In other embodiments, the control circuitry can present options to a user to enter values for both insulin and glucose to balance a pending reduced acute bolus.

In still other embodiments, a user can define present preferred levels associated with a percentage of remaining insulin budget. For example, at the beginning of an insulin budget term (e.g., day, days, week, weeks, month) where more than 95% of the insulin budget remains, a user can define a present where a glucose control level is set as “good” and the insulin control level is set as “poor.” Because there is sufficient insulin budget, the user may choose to keep relatively tight control of their glucose while freely withdrawing from their insulin budget. Similarly, toward the end of an insulin budget time period, or when less than 10% of the insulin budget remains, a user can enable the reduced acute bolus to be more aggressive at reducing insulin delivery thereby sacrificing glucose control to remain within the insulin budget. In preferred embodiments, the control circuitry will automatically prevent a user from over restricting insulin delivery at the expense of glucose control. Specifically, in preferred embodiments an input to the control circuitry can include a maximum time out of range of glucose. In these embodiments, regardless of insulin budget, the system will not allow an insulin delivery to be insufficient to drive the predicted glucose level below a predetermined level.

If operation 1016 is resolved by a user choosing to use non-insulin dependent techniques, operation 1024 allows the user to choose between exemplary behavior modification options that can modify the glucose response of a user. An exemplary technique includes operation 1026 that recommends behavior that improves the users glucose clearance or absorption. An exemplary behavior that could be recommended by operation 1026 may be associated with whether a user has indicated they will be ingesting carbohydrates. If the subject is planning to ingest carbohydrates, operation 1026 may recommend the user perform a physical activity for a minimum movement time within a defined window of time after ingesting the carbohydrates. Walking for 20-30 minutes after ingesting the carbohydrates is an exemplary, non-limiting physical activity that may be recommended. Other physical activities may be suggested where the objective is to improve glucose clearance in order to flatten or reduce the peak glucose value associated with the planned ingested carbohydrates. Operation 1026 may also recommend behavior that slows glucose absorption. An exemplary behavior that can slow glucose absorption is consuming foods that are high in fiber before consuming simple or processed carbohydrates.

An alternative exemplary technique to modify an anticipated glucose response is found in operation 1028 that recommends decreasing the amount of ingested carbohydrates. In many embodiments, operation 1028 can include referencing historical data from the data repository 118 for similar instances of current metabolic conditions relative to ingested carbs, dosed insulin and the resulting metabolic conditions. Additionally, operation 1028 can access the data repository 118 for similar physical state data in order to provide a recommendation for preferred ingested carbohydrates to stay within the insulin budget.

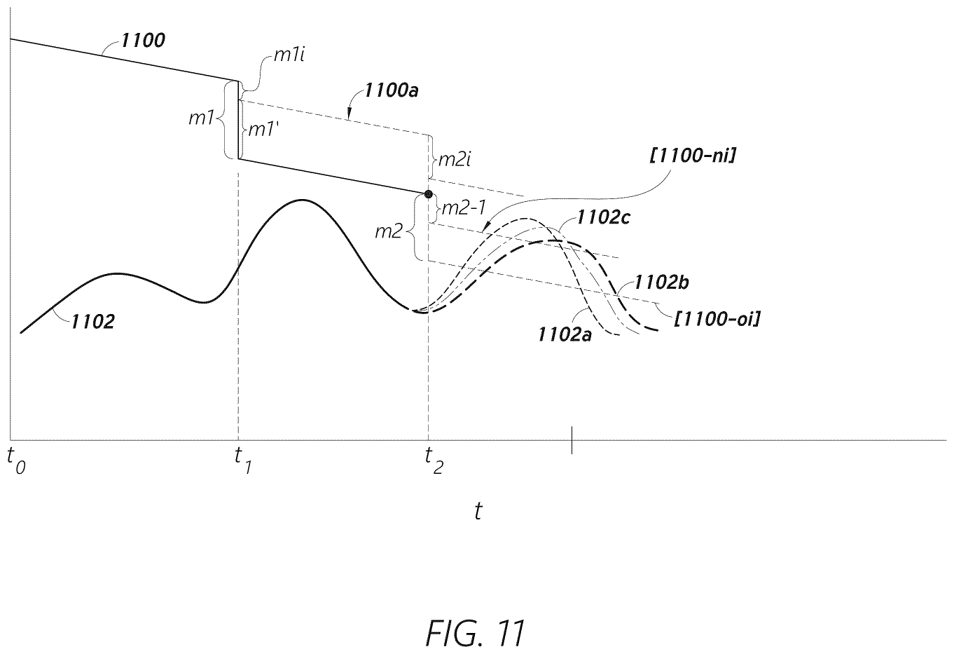

FIG. 11 is an exemplary illustration of remaining budget and analyte values with respect to time, in accordance with embodiments of the present invention. FIG. 11 includes budget trace 1100 that in some embodiments may be representative of aspects of an insulin budget. Analyte curve 1102, in many embodiments can represent at least one state of historical, real-time and projected analyte levels such as, but not limited to, glucose levels for a subject. For purposes of illustration and simplicity the time scale illustrated in FIG. 11 may be representative of the remaining budget and analyte levels or values for a 24 hour period of time, starting at midnight (or other time). In alternative embodiments, the time period may be for longer periods (e.g., multiple days, weeks, months, etc.) or shorter periods such as windows of time measured in minutes or hours. Additionally, in some embodiments, the windows of time can include some historical and projected periods of time (e.g., the previous two hours and projected forward two hours, or the previous 30 minutes and projected forward two hours, or combinations thereof).

In FIG. 11, historical data for both an insulin budget and glucose levels is shown as a solid line. Accordingly, the budget trace 1100 is shown steadily decreasing from t₀ until t₁, where t1 corresponds with a first meal for the subject. Similarly, the glucose level represented by the curve 1102 increases with the dawn effect and slowly decreases approaching t₁. At t₁, in anticipation of the first meal, insulin is delivered thereby reducing the insulin budget by m1. In accordance with some embodiments, an “ideal” budget 1100a can be displayed relative to the historical insulin budget. Additionally, an ideal insulin decrement m1i may also be displayed thereby visually illustrating the difference m1′ between m1 and m1i. Nevertheless, glucose levels for the subject increase and decrease between t₁ and t₂. At t₂, that represents the current time, the subject engages the acute module 910a of the insulin engine 910 (FIG. 10-1).

Based on inputs to the insulin engine, two projected insulin budgets are displayed. A first projected insulin 1100-ni is the anticipated remaining insulin budget determined by the insulin engine if the insulin budget is declinated by m2−1 and non-insulin dependent actions are used to modify the glucose response of the user. A second projected insulin 1100-oi is the anticipated remaining insulin budget determined by the insulin engine if the insulin budget is reduced by m2 and only insulin is used to address the planned anticipated meal. Also shown in FIG. 11 is the ideal budget 1100a and an ideal declination in the insulin budget m2i associated with a second meal.

The exemplary illustration in FIG. 11 further includes three projected glucose levels that correspond with different resolutions to resolve the insulin budget overdraft (operation 1016, FIG. 10-2). Projected or estimated glucose levels 1102a are associated with choosing to resolve the insulin budget overdraft by simply continuing to overdraft from the insulin budget. In the exemplary embodiment, estimated glucose levels 1102a substantially mimic or mirror the glucose response to the first meal. Estimated glucose levels 1102b and 1102c represent different non-insulin dependent behavior-based resolutions to the insulin budget overdraft. Projected glucose levels 1102b are based on a behavioral recommendation of increasing the amount of fiber ingested before eating the second meal. Increasing the fiber content before consuming carbohydrates can lower the peak glucose for a meal while also stretching or elongating the glucose response.

Projected glucose levels 1102c are based on a behavioral recommendation of a period of physical activity within 10 to 30 minutes of finishing the second meal. In many embodiments, the physical activity is walking at a moderate pace for 15 to 20 minutes after finishing the meal. The moderate physical activity enables cells, particularly muscles, to directly uptake glucose that is circulating in the bloodstream. The natural non-insulin dependent behavior of physical activity reduces the circulating glucose thereby reducing the peak glucose associated with the meal. This is reflected in estimated glucose levels 1102c where the peak glucose level is less than in peak glucose level 1102a.

The estimated glucose levels 1102b and 1102c illustrate how non-insulin dependent resolution of the insulin budget overdraft can result in projected glucose levels that result in reduced glucose time-in-range relative to projected glucose levels 1102a. Because the non-insulin dependent resolutions extend or prolong the glucose response curve, there may be additional time out of range relative to the only insulin resolution. However, insulin engine systems disclosed herein advantageously enable informed decisions to sacrifice a glucose control metric with respect to delivery of insulin. Specifically, as illustrated in FIG. 11, the glucose control metric is glucose time-in-range, typically associated with a subject's HbA1c, relative to insulin delivery.

For clarity, in FIG. 11 the insulin budget 1100 at t₂ is already below, or under, the ideal budget 1100 at t₂. However, in other exemplary situations, the insulin budget 1100 at t₂ may be slightly under or over budget where m2 will result in the projected insulin budget 1100-oi would result in an overdraft from the insulin budget while projected insulin budget 1100-ni would result in staying at or above the ideal budget 1100a, i.e., a situation where m2 is greater than m2i and m2−1 is less than m2i.

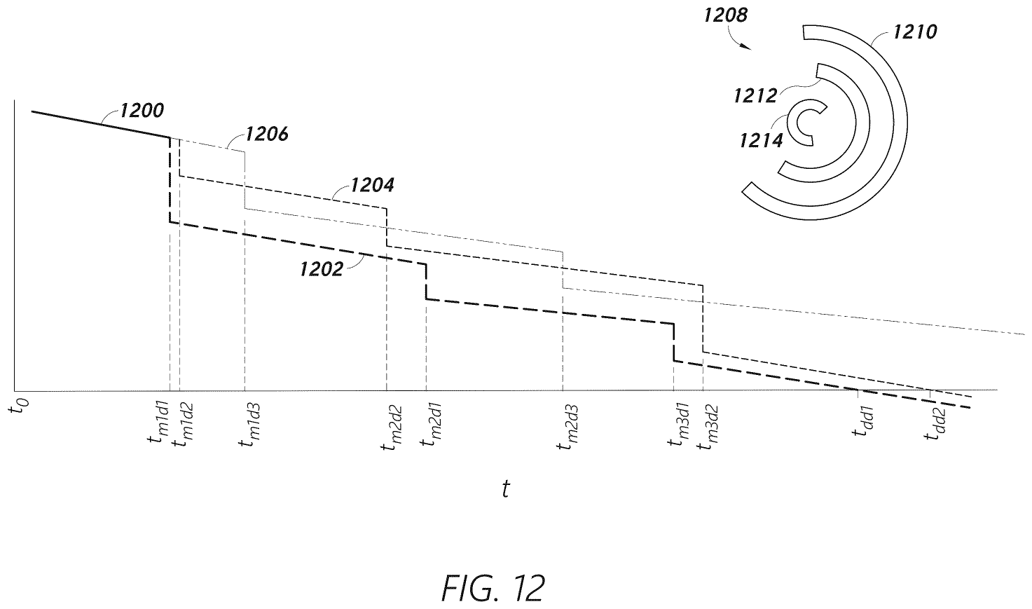

FIG. 12 is an exemplary illustration of multiple days of historical insulin budget data, in accordance with embodiments of the present invention. In FIG. 12 the initial budget at t₀ can correspond with a time such as, but not limited to midnight to allow the budget to encompass a full day. In other embodiments, time t₀ can correspond with any other time so long as the budget is for a 24-hour period. In FIG. 12, three days of insulin budget are shown. An initial budget of 1200 is shown for each day. FIG. 12 includes insulin budget traces for a first day 1202, a second day 1204, and a third day 1206. Additional exemplary data includes a time for a first meal. For example, t_{mld1} corresponds to the time for meal 1 on day 1. Similarly, t_{mld2} corresponds to the time for meal 1 on day 2. Likewise, t_{mld3} is the time when the subject consumed meal 1 on day 3. Similar times are indicated where indicators use a format of tm(X)d(Y) where t corresponds to a time, m(X) indicates a meal where (X) corresponds to the meal of the day and d(Y) indicates which day, where (Y) corresponds to the day. In some embodiments, the Y corresponds with a day of the month or a day of the week. Also shown are exemplary times t_{dd1} and t_{dd2}. Where t_{dd1} corresponds to time of budget deficit on day 1 and t_{dd2} corresponds to time of budget deficient on day 2. The use of meals to illustrate relatively large changes in the insulin budget should not be construed as limiting. The use of meals was to clearly illustrate the equivalent of a meal bolus. Additional bolus-like insulin delivery may occur throughout a designated time period for events such as small snacks, exercise, or even insulin delivery to correct an previous underestimation or carbohydrate intake. While these events are not shown, it should be understood that insulin delivery, irrespective of reason, will be decremented from the insulin budget.

An additional exemplary data display includes circular percentages 1208 that illustrate various aspects of data 1210, 1212 and 1214 determined by the system. Exemplary data represented by the circular percentages 1208 includes, but is not limited to daily insulin budget remaining, weekly insulin budget remaining, monthly insulin budget remaining, time within range for glucose, number of days exceeding insulin budget, risk score of developing iatrogenic hyperinsulinemia, percentage of time below insulin budget and the like.

The exemplary data shown in FIG. 12 should not be construed as limiting. In other embodiments additional data from the data repository 118 or other components or elements in the system can be displayed along with or overlaid with the historical insulin budget data. For example, in many embodiments, the insulin budget 1102 can be overlaid with the historical insulin budget data. Similarly, data such as, but not limited to plasma insulin, insulin resistance, or glucose disposal rate may optionally be shown in various embodiments of FIG. 12.

Throughout the description the system has been described as sensors associated with an electronics package that enable communication to additional devices. However, in some embodiments, the system 100 (FIG. 1) can be incorporated into a network of sensors that includes various combinations of multiple sensors (ranging from non-invasive, minimally invasive to invasive) that are optionally interconnected via an on-body network. The networked sensors can be similar to those described in U.S. patent application Ser. No. 15/417,055 filed on Jan. 26, 2017 and Ser. No. 16/054,649, filed on Aug. 3, 2018. Alternative embodiments utilize a variety of other sensors capable of measuring a variety of other conditions. Each different sensor within the networked sensors can be operated continuously or alternatively, periodically or episodically to gain additional insight into physiological states and conditions.

For example, while many embodiments disclosed above are specific to delivery of subcutaneous insulin, other embodiments may be configured to deliver a variety of infusion therapies such as those associated with nutrition, along with medications such as antibiotics, antiemetics, antifungals, antivirals, biologics, chemotherapy, corticosteroids, growth hormones, immunotherapy and the like, for the acute and/or chronic medical conditions to which they are associated. In some embodiments, metabolic data gathered by the implantable sensors can be used to improve or enhance chemotherapy treatment or provide insight regarding how well a subject is adhering to a prescribed diet. In other embodiments, sensors may be configured to detect a concentration of a

Accordingly, while the description above refers to particular embodiments of the invention, it will be understood that many modifications may be made without departing from the spirit thereof. In particular, while many embodiments are directed toward specific combinations of analytes and physical sensor data, it should be understood that where possible, each embodiment is capable of being combined with each and every other embodiment. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

Although certain preferred examples are disclosed above, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain spatially relative terms, such as “outer,” “inner,” “upper,” “lower,” “below,” “above,” “vertical,” “horizontal,” “top,” “bottom,” “distal,” “proximal,” and similar terms, are used herein to describe a spatial relationship of one device/element or anatomical structure to another device/element or anatomical structure. It should be understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

What is claimed is:

1. A multi-analyte sensor system comprising:
a sensor probe comprising:
a first set of electrodes including one or more first working electrodes configured to transduce glucose into electrical signals;
a second set of electrodes including one or more second working electrodes configured to transduce lactate into electrical signals; and
a third set of electrodes including one or more third electrodes that provide working and counter electrode functionality for the first set of electrodes and the second set of electrodes;
an electronics module configured to electrically interface with the sensor probe, the electronics module including a transceiver configured to transmit sensor data; and
control circuitry communicatively coupled to the electronics module and configured to:
determine a glucose state based on one or more signals from the first set of electrodes;
determine a lactate state based on one or more signals from the second set of electrodes; and
generate an insulin infusion pump control signal based on the one or more signals from the first set of electrodes and the one or more signals from the second set of electrodes.

2. The multi-analyte sensor system of claim 1, wherein the control circuitry is a component of the electronics module.

3. The multi-analyte sensor system of claim 1, wherein:
the control circuitry is further configured to determine a plasma insulin condition based on the one or more signals from the first set of electrodes and the one or more signals from the second set of electrodes; and
the insulin infusion pump control signal is based on the plasma insulin condition.

4. The multi-analyte sensor system of claim 1, wherein:
the control circuitry is further configured to determine an insulin sensitivity condition based on the one or more signals from the first set of electrodes and the one or more signals from the second set of electrodes; and
the insulin infusion pump control signal is based on the insulin sensitivity condition.

5. The multi-analyte sensor system of claim 1, wherein:
the sensor probe further comprises a fourth set of electrodes including one or more third working electrodes configured to transduce tissue oxygen into electrical signals; and
the insulin infusion pump control signal is based on one or more signals from the fourth set of electrodes.

6. The multi-analyte sensor system of claim 1, wherein:
the control circuitry is further configured to detect a meal intake state based on the glucose state; and
the insulin infusion pump control signal is based on the detected meal intake state and directs a bolus insulin dose.

7. The multi-analyte sensor system of claim 1, wherein:
the control circuitry is further configured to detect an exercise state based on the lactate state; and
the insulin infusion pump control signal is based on the detected exercise state and directs reduction in insulin delivery to prevent a hypoglycemia state.

8. The multi-analyte sensor system of claim 1, wherein:
the sensor probe is configured to detect tissue impedance; and
the insulin infusion pump control signal is based on the detected tissue impedance.

9. The multi-analyte sensor system of claim 1, further comprising an accelerometer associated with the electronics module, wherein the insulin infusion pump control signal is based on one or more signals from the accelerometer that indicate physical activity.

10. A continuous multianalyte monitoring system, comprising:
a skin-mounted sensor control unit, comprising:
a percutaneous multianalyte sensor including an insertion portion configured for transcutaneous positioning in a subcutaneous tissue of a user, the percutaneous multianalyte sensor being configured to sense levels of glucose and lactate in the subcutaneous tissue of the user; and
an adhesive patch disposed on a bottom surface of the skin-mounted sensor control unit and configured to adhere the skin-mounted sensor control unit to skin of the user;
a transceiver configured for wireless communication with the skin-mounted sensor control unit; and
control circuitry configured to:
receive and store signals from the percutaneous multianalyte sensor related to sensed levels of glucose and lactate;
determine a real-time insulin condition value based on the received signals related to sensed levels of glucose and lactate;
determine a metabolic health score based on the real-time insulin condition value; and
generate user interface data for rendering on a touch-interface display to visually display a graph of the glucose and lactate levels, the graph representing a first axis corresponding to time, a second axis corresponding to one or more of the glucose levels or lactate levels, and the metabolic health score.

11. The continuous multianalyte monitoring system of claim 10, wherein the control circuitry is further configured to maintain historical real-time insulin condition values in data storage of the control circuitry.

12. The continuous multianalyte monitoring system of claim 11, further comprising an automatic insulin delivery system configured to deliver basal insulin doses and bolus insulin doses based on at least one of the real-time insulin condition value or one or more of the maintained real-time insulin condition values.

13. The continuous multianalyte monitoring system of claim 12, wherein the control circuitry is further configured to determine a total insulin delivered by the automatic insulin delivery system based on basal insulin and bolus insulin delivered over a defined time period and determine a real-time insulin budget residual based on the total insulin.

14. The continuous multianalyte monitoring system of claim 10, wherein the real-time insulin condition value indicates a plasma insulin level of the user.

15. The continuous multianalyte monitoring system of claim 10, wherein the real-time insulin condition value indicates an insulin resistance condition of the user.

16. A multi-analyte sensor system comprising:
a sensor probe comprising:
a first set of working electrodes configured to transduce glucose into first electrical signals;
a second set of working electrodes configured to transduce lactate into second electrical signals; and
a third set of electrodes that are configured to provide reference and counter electrode functionality for the first set of working electrodes and the second set of working electrodes;
an electronics module configured to electrically interface with the sensor probe, the electronics module including a transceiver configured to wirelessly transmit sensor data; and
control circuitry communicatively coupled to the electronics module and configured to:
store an insulin budget value that indicates an insulin budget for administration to a user over a set period of time;
determine a glucose state based on one or more signals from the first set of working electrodes;
determine a lactate state based on one or more signals from the second set of working electrodes;
determine an insulin condition based on at least the glucose state and the lactate state;
determine an acute dose of insulin modification based on the insulin condition and an anticipated modified glucose state, and
provide recommendations for the insulin modification to reduce the acute dose based on a residual of the insulin budget.

17. The multi-analyte sensor system of claim 16, wherein the insulin modification is a change in basal insulin delivery.

18. The multi-analyte sensor system of claim 17, wherein the change in basal insulin delivery is a suspension of basal insulin delivery.

19. The multi-analyte sensor system of claim 16, wherein the insulin modification is an acute dose of insulin and the recommendations to reduce the acute dose is provided if the acute dose of insulin exceeds a projected residual of the insulin budget.

20. The multi-analyte sensor system of claim 19, wherein the recommendations include insulin dependent and non-insulin dependent recommendations to reduce the acute dose.

21. The multi-analyte sensor system of claim 20, wherein the insulin dependent recommendations provide an option to favor closer adherence to the insulin budget rather than a recommendation to improve glucose time-in-range.

22. The multi-analyte sensor system of claim 20, wherein the insulin dependent recommendations provide an option to favor close adherence to glucose time-in-range and exceeds the insulin budget.

23. The multi-analyte sensor system of claim 22, wherein non-insulin dependent recommendations include behavior modifications that (i) reduce anticipated meal metrics that require insulin, or (ii) improve glucose clearance, or (iii) slow glucose absorption.
